(19) United States
(12) Patent Application Publication
(10) Pub. No.: US 2025/0367134 A1
(43) Pub. Date: Dec. 4, 2025

(54) PERIPHERALIZATION OF CENTRALLY-ACTING CANNABINOID-1 RECEPTOR ANTAGONISTS BY NANOPARTICLES FOR THE TREATMENT METABOLIC RELATED CONDITIONS

(71) Applicant: Yissum Research Development Company of the Hebrew University of Jerusalem Ltd., Jerusalem (IL)

(72) Inventors: Joseph TAM, Jerusalem (IL); Gershon GOLOMB, Efrat (IL); Shira HIRSCH, Jerusalem (IL); Meital NAIM, Jerusalem (IL)

(73) Assignee: Yissum Research Development Company of the Hebrew University of Jerusalem Ltd., Jerusalem (IL)

(21) Appl. No.: 18/875,208

(22) PCT Filed: Jun. 14, 2023
(86) PCT No.: PCT/IL2023/050611

§ 371 Date: Dec. 16, 2024

Related U.S. Application Data
(60) Provisional application No. 63/366,545, filed on Jun. 17, 2022.

(51) Int. Cl. A61K 9/51; A61K 9/1271; A61K 31/454

(52) U.S. Cl. CPC A61K 9/5153; A61K 9/1271; A61K 31/454

(57) ABSTRACT

The technology includes selective modulation of only a peripheral CB1R by using a CB1R antagonist contained in a peripherally restricted delivery system.

### TECHNOLOGICAL FIELD

The invention generally contemplates CB1 receptor antagonists and nanoparticles forms thereof.

### BACKGROUND OF THE INVENTION

Non-alcoholic fatty liver disease (NAFLD), a common and potentially serious condition often associated with obesity, is a major cause of morbidity and mortality. It is characterized by a spectrum of liver conditions, ranging from an ectopic accumulation of fat in the liver (hepatic steatosis), to non-alcoholic steatohepatitis (NASH), which can be complicated by fibrosis, cirrhosis, end-stage liver failure, and hepatocellular carcinoma (HCC). Several lines of evidence suggest that NAFLD promotes type 2 diabetes (T2D). Although NAFLD is present in 20-30% of the general population, it reaches the impressive prevalence of 50-75% in patients affected by T2D. Once T2D is fully developed, it further contributes not only to the development of steatosis but also to NASH, fibrosis, cirrhosis, and possibly HCC. Therefore, early therapeutic interventions are imperative for the treatment of NAFLD patients at risk for developing T2D.

Recent findings have revealed the significant role played by the endocannabinoid (eCB) system in the pathogenesis of T2D and NAFLD. eCBs are endogenous lipid ligands that interact with the cannabinoid receptors, CB1R and CB2R, which also recognize Δ9-tetrahydrocannabinol (THC), the psychoactive component of marijuana, and mediate its biological effects. By activating CB1Rs, eCBs increase the appetite (the ‘munchies’) and lipogenesis in adipose tissue and liver as well as induce insulin resistance and dyslipidemia. In addition, elevated circulating levels of eCBs have been reported in obese patients vs. lean controls; they are positively associated with waist circumference, body mass index (BMI), visceral adiposity, insulin resistance, and NAFLD. Thus, an overactive eCB/CB1R system contributes to the development of visceral obesity, hepatic steatosis, T2D, and other medical complications.

Consequently, pharmaceutical companies have been encouraged to develop drugs that block CB1Rs as a potential treatment for these clinical conditions. The first such compound, rimonabant (SR141716, Acomplia®, Sanofi-Aventis), was found effective, not only in reducing body weight in obese and overweight individuals, but also in ameliorating the associated metabolic abnormalities, including hepatic steatosis, insulin resistance, and T2D. However, the neuropsychiatric side effects, including depression, anxiety, and suicidal ideation, led to its worldwide withdrawal as a viable medicine in 2009, and halted further therapeutic development of this class of compounds. In addition, preclinical evidence has emerged indicating that CB1R in peripheral tissues is mostly involved in hormonal and metabolic regulation. This raised the prospect that selective targeting of peripheral CB1R may retain some or most of its metabolic benefits while avoiding any neuropsychiatric liability.

A recent study provided comprehensive evidence for the therapeutic potential of targeting the peripheral CB1R for the treatment of obesity, T2D, NAFLD, chronic kidney disease, and osteoporosis [1-9].

### REFERENCES

[1] Azar, S., et al., Reversal of diet-induced hepatic steatosis by peripheral CB1 receptor blockade in mice is p53/miRNA-22/SIRT1/PPARalpha dependent. Mol Metab, 2020. 42: p. 101087.
[2] Drori, A., et al., CB1R regulates soluble leptin receptor levels via CHOP, contributing to hepatic leptin resistance. Elife, 2020. 9.
[3] Drori, A., et al., Cannabinoid-1 receptor regulates mitochondrial dynamics and function in renal proximal tubular cells. Diabetes Obes Metab, 2019. 21(1): p. 146-159.
[4] Hinden, L., et al., Modulation of Renal GLUT2 by the Cannabinoid-1 Receptor: Implications for the Treatment of Diabetic Nephropathy. J Am Soc Nephrol, 2018. 29(2): p. 434-448.
[5] Knani, I., et al., Targeting the endocannabinoid/CB1 receptor system for treating obesity in Prader-Willi syndrome. Mol Metab, 2016. 5(12): p. 1187-1199.
[6] Tam, J., et al., Peripheral cannabinoid-1 receptor inverse agonism reduces obesity by reversing leptin resistance. Cell Metab, 2012. 16(2): p. 167-79.
[7] Tam, J., et al., Peripheral CB1 cannabinoid receptor blockade improves cardiometabolic risk in mouse models of obesity. J Clin Invest, 2010. 120(8): p. 2953-66.
[8] Udi, S., et al., Proximal tubular cannabinoid-1 receptor regulates obesity-induced CKD. J Am Soc Nephrol, 2017. 28(12).
[9] Baraghithy, S., et al., Renal Proximal Tubule Cell Cannabinoid-1 Receptor Regulates Bone Remodeling and Mass via a Kidney-to-Bone Axis. Cells, 2021. 10 (2).

### GENERAL DESCRIPTION

In view of the pivotal role of cannabinoid type 1 receptor (CB1R) in the pathogenesis of NAFLD and T2D, targeting the diseased activated CB1Rs in the liver seems an attractive strategy to stop or reverse the progression of these conditions. In that sense, nanomedicine offers a feasible approach to target centrally acting insoluble lipophilic drugs, like rimonabant, directly to the liver while reducing its centrally mediated adverse effects. To meet the challenges associated with the development of delivery methodologies that prevent brain-blood barrier transport of active agents, the inventors of the technology disclosed herein have developed a delivery platform that is exemplified by poly(lactic-co-glycolic acid) (PLGA)-encapsulated rimonabant nanoparticles (Rimo-NPs). The unique delivery system of the invention has demonstrated effective inhibition of hepatic CB1R, improving obesity-induced hepatic steatosis, dyslipidemia, and insulin resistance, without apparent neuropsychiatric liability.

In other words, the invention contemplates selective modulation of only the peripheral CB1R by using a CB1R antagonist contained in a peripherally restricted delivery system.

Thus, in a first of its aspects, the invention concerns a peripherally restricted nanocarrier comprising a cannabinoid 1 receptor (CB1R) antagonist.

The invention further provides a peripherally restricted nanocarrier comprising a CB1R antagonist, for selective modulation of a peripheral CB1R.

Further provided is a nanocarrier comprising a CB1R antagonist, wherein said nanocarrier is configured for peripheralization of the CB1R antagonist, without inducing a CNS effect.

In some embodiments, the nanocarrier is an acid-terminated or an ester terminated PLGA having a molecular weight between 30 and 100 kDa. In some embodiments, the CB1R antagonist is a central nervous system (CNS) active CB1R antagonist.

As disclosed further below, nanocarriers of the invention are suitable for treatment or prevention of non-alcoholic fatty liver disease (NAFLD) or type 2 diabetes (T2D) or dyslipidemia, wherein said nanocarrier is configured for peripheralization of the CB1R antagonist, without inducing a CNS effect. In some embodiments, the antagonist is a central nervous system (CNS) acting CB1R antagonist, which would otherwise induce a CNS effect.

The invention further provides a nanocarrier comprising a central nervous system (CNS) acting CB1R antagonist suitable for treatment or prevention of non-alcoholic fatty liver disease (NAFLD) or type 2 diabetes (T2D) or dyslipidemia, wherein said nanocarrier is configured for peripheralization of the CNS acting CB1R antagonist, without inducing a CNS effect.

As disclosed herein, the technology concerns the ability to selectively modulate a peripheral effect without inducing a CNS effect expected from systemic administration of such active agents as CNS acting CB1R antagonists. By limiting the effect of the CNS acting antagonist to only the peripheral CB1R, side effects associated with CNS modulation are diminished or substantially reduced. These side effects may be psychiatric side effects and others. The ability to reduce or diminish such side effects is achieved by containment of the CB1R antagonist in a peripherally restricted nanocarrier. The “peripherally restricted nanoparticulate carrier” is any such nanocarrier, which may be a nanoparticle, a nanocapsule or generally a nanoparticulate carrier less than about 1 micron in diameter. The “nanocarrier” is a carrier structure, different from a liposome, which is biocompatible with and sufficiently resistant to chemical and/or physical destruction by the environment of use such that a sufficient amount of the nanocarriers remains substantially intact after administration. The nanocarrier may be in the form of injectable carriers, which exhibit no effective transport capabilities through the blood-brain barrier (BBB), or which crossing the BBB is restricted or prevented in view of the nanocarriers' size, polarity and hydrophobicity. The selectively permeable blood-brain barrier prevents the efficient transfer of high molecular weight drugs from the blood to the brain parenchyma and thus hinders effective CNS involvement. As only lipid soluble (lipophilic) molecules with a low molecular weight (under 400-600 Da) and positive charge can cross the BBB, nanocarriers of the invention have been configured to adopt hydrophilic or poorly hydrophobic characteristics as well as neutral or negative surface charges. Thus, nanocarriers as used herein are of a size (and/or molecular weight), polarity and hydrophobicity that “prevent their effective transport through the blood-brain barrier (BBB)” or in other words, which substantially reduces or diminishes the effect of the CNS active antagonist on the central nervous system, thereby eliminating or reducing associated side effects.

In some embodiments, the nanocarriers are of an average size that is less than 500 nm in size (e.g., in diameter), or are of an average size between 80 and 500 nm. In some embodiments, the nanocarriers are of an averaged size (e.g., diameter) between 80 and 500 nm, 80 and 450 nm, 80 and 400 nm, 80 and 350 nm, 80 and 300 nm, 80 and 250 nm, 80 and 200 nm, 80 and 150 nm, 90 and 500 nm, 80 and 450 nm, 90 and 400 nm, 90 and 350 nm, 90 and 300 nm, 90 and 250 nm, 90 and 200 nm, 100 and 500 nm, 100 and 400 nm, 100 and 300 nm, or 100 and 200 nm.

In some embodiments, the nanocarriers are of an average size that is between 100 and 300 nm.

In some embodiments, the nanocarriers are formed of a naturally occurring, synthetic or semi-synthetic polymer having a molecular weight below 600 kDa. The polymer is a stable, biodegradable polymer material that may be made of random copolymers or block copolymers. The nanocarriers typically carry a neutral or a negative surface charge. In some embodiments, the nanocarriers have a zeta potential of between around zero to minus 1. In other embodiments, the particles have a zeta potential that is between −1 (minus 1) and −10 (minus 10) mV. In other embodiments, the particles have a zeta potential that is between −8 and −10.

The nanocarriers are hydrophilic in nature or poorly hydrophobic. In some embodiments, the nanocarriers are formed of a biodegradable hydrophobic polymer. In other examples, the nanocarriers may be formed of a hydrophilic polymer. The polymer may be neutral or charged, thereby ensuring increased hydrophilic behavior, and a lesser susceptibility for BBB crossing. In some embodiments, the nanocarriers are formed of a biodegradable polymer having surface functionalities increasing its hydrophilic characteristics. In some embodiments, such functionalities may be hydroxyl groups, carboxylic acid groups, ester groups, amines, and others capable of hydrogen bonding.

Non-limiting examples of polymeric nanocarriers include acid or ester substituted or terminated polymers such as dextran, carboxymethyl dextran, chitosan, trimethylchitosan, poly(lactic-co-glycolic acid) (PLGA), polylactic acid (PLA), polyglycolic acid (PGA), polyanhydrides, polyacrylates, polymethacrylates, polyacylamides, polymethacrylate, polycaprolactone (PCL), poly (α-hydroxy acids), polyhydroxyalkanoates (PHAs), poly (lactones), and poly(alkyl cyanoacrylates) (PACA), PLGA, poly(D,L-lactide-co-glycolide), poly(D,L-lactide), poly(D,L-lactide-co-lactide), poly(L-lactide), poly(glycolide), poly (L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(esteramide), poly(ester-sulfoester amide), poly(orthoester), poly(anhydride), and polysaccharides, such as alginate and chitosan, poly-ε-(D, L-lactide-co-caprolactone) (PLCL), LGA-PEG block co-polymers, human serum albumin (HSA), gelatin, hyaluronic acid and derivatives, Poloxamer (Pluronics) and others.

In some embodiments, the nanocarrier is formed of a mixture of polymers or two or more polymers, as defined herein. In some embodiments, the two or more polymers are selected to modify at least one property of the nanocarrier. For example, a polymer mixture may include PEG for increasing hydrophilicity for enhanced release rate of the active or a Poloxamer (Pluronics) for modifying the viscosity/enhanced release.

In some embodiments, the nanocarriers are formed of PLGA nanoparticles.

In some embodiments, the nanocarriers are formed of a polymeric mixture comprising PLGA.

In some embodiments, the nanocarriers are formed of a polymeric matrix comprising or consisting PLA, PGA and/or PLGA.

In some embodiments, the nanocarrier is formed of an acid-terminated PLGA, namely a PLGA having end carboxylic acid functionalities.

In some embodiments, the nanocarriers comprise a plurality of different nanocarrier types, e.g., made of different polymeric materials. In some embodiments, at least one of said nanocarrier types is a nanocarrier population made of PLGA.

The polymer making up nanocarriers of the invention may be of any low molecular weight. For purposes herein, the polymer is typically of a molecular weight between 20 KDa and 400 kDa. In some embodiments, the polymer molecular weight is between 50 and 400 kDa, 100 and 400 kDa, 50 and 200 kDa, 50 and 100 kDa, or the molecular weight is 50 kDa, 60 kDa, 70 kDa, 80 kDa, 90 kDa or 100 kDa.

In some embodiments, the polymer is PLGA of a molecular weight between 50 and 100 kDa.

In some embodiments, the PLGA is functionalized with acid or ester groups.

In some embodiments, the nanocarriers are PLGA nanocarriers, wherein the PLGA having a molecular weight between 50 kDa and 100 kDa and is formed of a lactic acid to glycolic acid ratio of 50:50 (or 1:1). In some embodiments, the lactic acid to glycolic acid ratio may be 75:25, 65:35, 85:15 or any other ratio.

In some embodiments, the PLGA nanocarriers are formed of a PLGA wherein the lactic acid to glycolic acid ratio may be between 3:1 to 1:1, or between 3:1 and 1:3.

As known in the art, the PLGA copolymer may be synthesized or obtained commercially. Generally, PLGA may be synthesized via random ring-opening copolymerization of lactic acid and glycolic acid by ester linkages. Different ratios of glycolic acid to lactic acid can be selected, e.g., 50:50 and 75:25 lactic acid to glycolic acid. The ratio between the two monomers may be tailored for achieving desired final properties of the nanoparticle. The amount of each monomer may determine the hydrophobicity of the polymer, the rate of degradation and its structure. The PLGA may be terminated in a carboxyl group (acid-terminated) or in an alkyl ester (ester-terminated). The alkyl ester may vary based on the reaction conditions and precursors used. The alkyl ester may comprise between 1 carbon to several dozen carbon atoms.

Both the acid-terminated and the ester-terminated PLGA polymers may be commercially obtained.

In some embodiments, the nanocarrier is formed of an acid-terminated or an ester terminated PLGA, having a molecular weight between 30 and 100 kDa.

In some embodiments, the nanocarrier is formed of an acid-terminated PLGA having a molecular weight of 50 kDa.

In some embodiments, the nanocarrier is formed of an ester-terminated PLGA having a molecular weight of 70 kDa.

In some embodiments, PLGA is provided in combination with PLA.

In some embodiments, the PLGA comprising a lactide-glycolide ratio of 50:50 and a molecular weight between 30 kDa and 100 kDa.

In some embodiments, the invention further provides a peripherally restricted nanocarrier comprising a central nervous system (CNS) acting CB1R antagonist, for selective modulation of a peripheral CB1R, wherein the nanocarrier is PLGA nanocarrier having a size between 100 and 300 nm and a zeta potential between zero and −10 (minus 10) mV.

In some embodiments, the PLGA is of a molecular weight between 50 and 100 kDa.

In some embodiments, a peripherally restricted nanocarrier is provided that comprised a CB1R antagonist, e.g., a central nervous system (CNS) acting CB1R antagonist, wherein the nanocarrier is a PLGA nanocarrier that is optionally formed of an acid or ester-terminated PLGA, wherein the nanocarrier having a size between 100 and 300 nm, a zeta potential between zero and −10 (minus 10) mV and a molecular weight of between 50 and 100 kDa.

The type of nanocarrier used according to the invention may be selected based on one or more considerations known in the art. Irrespective of whether a nanocapsule is used or any other type of a nanoparticle, the active material may be carried by or contained within the nanocarriers to achieve effective release therefrom. The term “carried by” or “contained in” encompass any mode of association between the nanocarriers and the CB1R antagonist, which may be generally contained within the nanocarrier or associated to their surfaces. Such an association may include, without limitation, impregnation, encapsulation, or containing within the nanocarriers; or within the matrix material making up the nanocarriers, e.g., within the polymeric material; or adsorption to the surface of the nanocarrier; or attachment to the surface of the nanocarriers by physical or chemical associations such as hydrophobic, hydrostatic, hydrogen bonds, or any association allowing for release of the antagonists therefrom. The release may be by way of cleavable bonds, active release, passive release or any decomposition means involving decomposition of the nanocarriers.

In some embodiments, the antagonist is carried within the nanocarrier. In some embodiments, the antagonist is not associated with a surface region of the nanocarrier. In some embodiments, the nanocarrier does not comprise oleylcysteinamide (OCA).

The “CB1R antagonist” is a receptor blocker or a reverse agonist, which in most general terms partially or fully blocks, inhibits, or neutralizes a biological function of a peripheral CB1 receptor. By partially or fully blocking, inhibiting, or neutralizing a biological function of the receptor, prevention or treatment of a variety of metabolic syndromes can be achieved. These metabolic syndromes may include obesity, insulin resistance, diabetes, coronary heart disease, fatty liver, hepatic cirrhosis, chronic kidney disease and cancer. The “CNS-acting CB1R antagonist” is any therapeutically active agent that is a neutral antagonist or inverse agonist of the CB1R receptor, that without being contained in the nanocarrier of the invention would have, upon administration, crossed the blood-brain barrier and caused central mediated side effects. The antagonist is not a natural plant cannabinoid or a naturally occurring endocannabinoids, known or realized to act as a CNS-acting CB1R antagonist.

In some embodiments, the antagonist, e.g., a CNS-acting antagonist, is an agent capable of effective modulation (improvement or prevention or treatment) of a metabolic disease or pathology such as NAFLD.

In some embodiments, the antagonist, e.g., CNS-acting antagonist, is an agent capable of effective modulation (improvement or prevention or treatment) of a metabolic disease or pathology, including for example obesity-induced dyslipidemia, hepatic steatosis, liver injury, insulin resistance, reversion of liver weight, elevated hepatic triglyceride content, hepatocyte ballooning, fat accumulation, hepatocellular damage, improvement of insulin sensitivity, as reflected by their ability to reduce glucose levels following a bolus of insulin, reduction of hyperinsulinemia, chronic kidney disease, diabetes, hypertension, and improvement of Homeostatic Model Assessment for Insulin Resistance (HOMA-IR).

In some embodiments, the antagonist, e.g., CNS-acting CB1R antagonist, is an anorectic drug that reduces appetite, thereby leading to weight loss, while inducing substantial side effects (such as psychiatric side effects) when administered in a way different from the disclosed herein.

In some embodiments, the antagonist is rimonabant or an analogue or isostere or a derivative thereof.

Rimonabant, N-(piperidin-1-yl)-5-(4-chlorophenyl)-1-(2,4-dichlorophenyl)-4-methyl-1H-pyrazole-3-carboxamide hydrochloride), having the structure

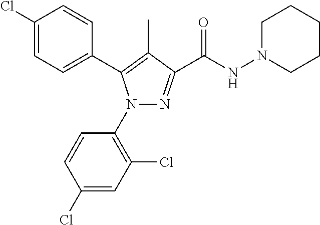 [SMILES: Cc1c(C(=O)NN2CCCCC2)nn(-c2ccc(Cl)cc2Cl)c1-c1ccc(Cl)cc1]

is an anorectic anti-obesity drug that was first approved in 2006 but was withdrawn due to serious psychiatric side effects. It is an inverse agonist for CB1R and was the first drug approved in that class. As demonstrated herein, PLGA-encapsulated rimonabant nanoparticles demonstrated effective inhibition of hepatic CB1R, improving obesity-induced hepatic steatosis, dyslipidemia, and insulin resistance, without apparent neuropsychiatric side effects.

Additional CNS-acting CB1R antagonists are analogues and isosters of rimonabant, as known as specified for example in Foloppe, N.; Allen, N. H.; Bentlev, C. H.; Brooks, T. D.; Kennett, G.; Knight, A. R.; Leonardi, S.; Misra, A.; et al. (2008), “Discovery of novel class of selective human CB1 inverse agonists”, Bioorganic & Medicinal Chemistry Letters, 18 (3): 1199-1206, which content is herein incorporated by reference.

In some embodiments, the antagonist is Surinabant (SR147778) of the structure

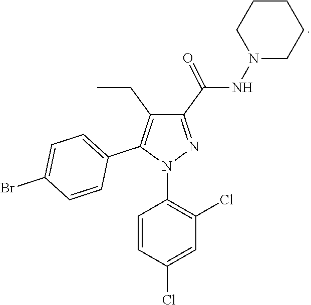 [SMILES: CCc1c(C(=O)NN2CCCCC2)nn(-c2ccc(Cl)cc2Cl)c1-c1ccc(Br)cc1]

In some embodiments, the antagonist is Ibipinabant (SLV-319) having the structure

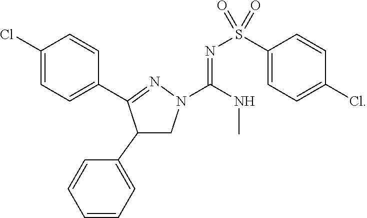 [SMILES: CN/C(=N\S(=O)(=O)c1ccc(Cl)cc1)N1CC(c2ccccc2)C(c2ccc(Cl)cc2)=N1]

In some embodiments, the antagonist is Otenabant (CP-945,598) of the structure

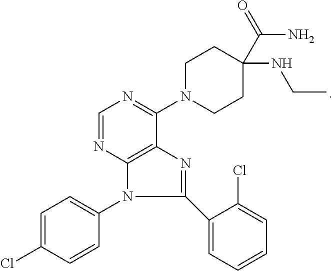 [SMILES: CCNC1(C(N)=O)CCN(c2ncnc3c2nc(-c2ccccc2Cl)n3-c2ccc(Cl)cc2)CC1]

In some embodiments, the antagonist is Drinabant (AVE-1625) of the structure

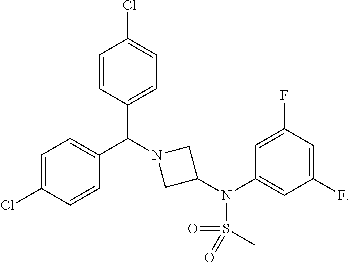 [SMILES: CS(=O)(=O)N(c1cc(F)cc(F)c1)C1CN(C(c2ccc(Cl)cc2)c2ccc(Cl)cc2)C1]

In some embodiments, the antagonist is of structure:

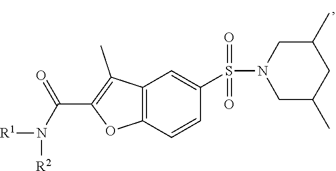

wherein R1 may be H or a C1-C6alkyl, such as methyl, ethyl, propyl, butyl, pentyl and hexyl, and R2 may be a substituted or an unsubstituted C6-C10 aryl, C3-C10carbocyclyl, C5-C10heteroaryl, C1-C6alkyl, C2-C6alkenyl, C2-C6alkynyl and others. Non-limiting examples of R2 include benzyl and substituted benzyl, pyridyl, chlorinated phenyl, dimethylphenyl, and others.

Any of the antagonists disclosed herein, may be known in the art.

The present invention further concerns a composition or a pharmaceutical composition comprising an antagonist, e.g., a CNS-acting CB1R antagonist, contained in peripherally restricted nanocarriers.

Compositions or pharmaceutical compositions of the invention may comprise a single population of nanocarriers or a plurality of different nanocarriers. Where a plurality of populations is concerned, each nanocarrier population may differ from another in the material from which the nanocarriers are formed, the nanocarriers size, the active antagonist contained in the nanocarriers, etc. To ensure stability and continuous integrity of the nanocarriers, the composition may be formed in a carrier that maintains the integrity of the nanocarriers. The carrier may be a solid carrier or a liquid carrier that does not dissolve or cause decomposition of the nanocarrier material. The carrier is typically one which does not cause or induce leaching out of the antagonist from the nanocarriers.

In some embodiments, the composition is a solid composition, wherein the nanocarriers are carried in a solid carrier (dispersed or as a solid solution), or a liquid composition which may be in a form of a dispersion or a suspension.

Compositions of the invention may be prepared by any method known in the art. In general, such methods include bringing the nanocarriers into association with a carrier or excipient, and/or one or more other accessory ingredients, and subsequently shaping, and/or providing the composition into a desired single- or multi-dose unit, or into a formulation for a variety of forms of administration. Relative amounts of the active ingredient, the excipient, and/or any additional ingredients in a composition described herein may vary.

Different stabilizers and preservatives may be used. Such may include suspending agents, cryoprotectants, antioxidants, chelating agents, antimicrobial preservatives, antifungal preservatives, antiprotozoan preservatives, alcohol preservatives, acidic preservatives, and other preservatives. In some embodiments, the preservative is an antioxidant. In some embodiments, the preservative is a chelating agent.

Compositions of the invention may be tailored for any type of administration to a human or animal subjects. Typically, compositions of the invention are suitable for parenteral administration, and thus may include aqueous and non-aqueous carriers, or may be in a form of an isotonic sterile injection solution, which can contain anti-oxidants, buffers, bacteriostats, and solutes that render the composition isotonic with the blood of the intended recipient, and aqueous and non-aqueous sterile suspensions that include cryoprotectants, suspending agents, solubilizers, thickening agents, stabilizers, and preservatives. The nanocarriers may be administered in a physiologically acceptable pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethylene glycol), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral compositions include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-aminopropionates, and 2-alkyl-imidazoline quaternary ammonium salts, and (3) mixtures thereof.

The parenteral compositions can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions may be prepared from sterile solids such as powders, granules, and tablets of the kind previously described.

The nanocarriers of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), Handbook on Injectable Drugs, 20th edition Twentieth Edition by American Society of Health-System Pharmacists; and add: ASHP Injectable Drug Information 2023.

In some embodiments, compositions containing nanocarriers of the invention are adapted for administration via subcutaneous (sc), intramuscular (im), intravenous (iv), intraperitoneal (ip) administration or via laparoscopy.

Also provided is use of a nanocarrier comprising a CB1R antagonist, e.g., a CNS-active CB1R antagonist, in a method of peripheralization of the CNS acting CB1R, without inducing a CNS effect.

In some embodiments, the method is for treating a metabolically-associated disease or pathology.

The invention further provides a method for treating a metabolically associated disease or pathology, the method comprising administering to a subject in need of such treatment a therapeutically effective amount of a CB1R antagonist, e.g., a CNS-active CB1R antagonist contained in peripherally restricted nanocarriers, wherein treatment does not involve CNS derived side effects.

The invention further provides a method of improving a condition of a subject suffering from a metabolically-associated disease or pathology, without inducing CNS derived side effects, the method comprising administering to said subject a composition comprising nanocarriers containing a CB1R antagonist, e.g., a CNS-active CB1R antagonist.

The invention further provides a method of peripheralization of a CNS-active CB1R antagonist without inducing CNS-derived side effects, the method comprising containment of said CNS-active CB1R antagonist in nanocarriers and administering said nanocarriers to the subject.

Also provided is a method of treating or preventing a non-alcoholic fatty liver disease (NAFLD) or type 2 diabetes (T2D) or dylipidemia in a subject, the method comprising administering to said subject a composition comprising nanocarriers comprising a central nervous system (CNS) acting CB1R antagonist suitable for treatment or prevention of the NAFLD or T2D, wherein said nanocarrier is configured for peripheralization of the CNS acting CB1R antagonist, without inducing a CNS effect.

As disclosed herein, the antagonist, e.g., the CNS-active CB1R antagonist, is an anorectic or an anti-obesity drug that induced CNS-derived side effects such as psychiatric effects, cardiac affects and others. When contained in nanocarriers of the invention, the nanocarriers allow the antagonist to maintain its effective anorectic or anti-obesity effect while reducing or diminishing the inherent CNS-derived side effects. These side effects are completely reduced or abolished or are reduced by at least 90% (wherein 9 out of 10 subjects do not suffer from CNS-derived side effects or the vast majority of the side effects are not observed) or 95% or 99%.

Compositions of the invention may comprise an “effective amount” of the nanocarriers that is determined based on the amount of the antagonist carried thereby. The effective amount may be determined by such considerations as may be known in the art. The amount must be effective to achieve the desired therapeutic effect as described herein, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

Antagonists used according to the invention are used to achieve an improvement in a subject's medical condition, typically a metabolically-associated disease or pathology; or in preventing or treating such a disease or a pathology. Thus, the term “treatment” encompasses administering of a therapeutic amount of a composition of the invention which is effective to ameliorate undesired symptoms associated with a disease or pathology, to prevent the manifestation of symptoms before they occur, to slow down progression of the disease or pathology, slow down deterioration of symptoms, enhance onset of remission period, slow down irreversible damage caused in the progressive chronic stage of the disease or pathology, to delay onset of said progressive stage, to lessen severity or cure the disease, to improve survival rate or more rapid recovery, or to prevent the disease or pathology form occurring or a combination of two or more of the above.

In some embodiments, the metabolic disease or pathology is a non-alcoholic fatty liver disease (NAFLD). The NAFLD refers to fatty liver conditions in which there is no history of alcohol consumption or in which alcohol consumption is not related to the occurrence of liver steatosis. The fatty liver condition refers to an abnormal accumulation of triglyceride in liver cells, compared to normal levels of triglyceride. The fatty liver may be caused by a lipid metabolism disorder or a defect in the process of carrying excessive fat in the liver cells, and is mainly caused by disorders of lipid metabolism in the liver.

NAFLD may be categorized into primary and secondary non-alcoholic fatty liver diseases depending on the pathological cause. The primary one is caused by hyperlipidemia, diabetes, obesity or the like which is a characteristic of metabolic syndrome. The secondary one is a result of nutritional causes (sudden body weight loss, starvation, intestinal bypass surgery), various drugs, toxic substances (poisonous mushrooms, bacterial toxins), metabolic causes and other factors.

In some embodiments, the NAFLD includes non-alcoholic fatty liver, non-alcoholic steatohepatitis (NASH), cirrhosis, and liver cancer.

In some embodiments, the metabolic disease or pathology is one or more of metabolic syndrome, obesity-induced dyslipidemia, hepatic steatosis, liver injury, insulin resistance, reversion of liver weight, elevated hepatic triglyceride content, hepatocyte ballooning, fat accumulation, hepatocellular damage, improvement of insulin sensitivity, as reflected by their ability to reduce glucose levels following a bolus of insulin, reduction of hyperinsulinemia, and improvement of Homeostatic Model Assessment for Insulin Resistance (HOMA-IR).

In some embodiments, the disease is dyslipidemia, hepatic steatosis, fatty liver, type-2 diabetes, insulin resistance, chronic kidney disease, and obesity.

In some embodiments, the disease is obesity.

In some embodiments, the metabolic associated disease or pathology is manifested by an increase in liver weight, elevated hepatic triglyceride content, hepatocyte ballooning, fat accumulation, hepatocellular damage, decreased insulin sensitivity, hyperinsulinemia, reduced body weight, ameliorated type-2 diabetes, or prevented chronic kidney disease.

### the Invention Provides:

A nanocarrier comprising a cannabinoid 1 receptor (CB1R) antagonist, wherein said nanocarrier is configured for peripheralization of the CB1R antagonist, without inducing a CNS effect, and wherein the nanocarrier is an acid-terminated or an ester terminated PLGA having a molecular weight between 30 and 100 kDa.

In some configurations of any of the nanocarriers according to the invention, the nanocarrier is for use in a method of treatment or prevention of non-alcoholic fatty liver disease (NAFLD) or type 2 diabetes (T2D) or dyslipidemia, wherein said nanocarrier is configured for peripheralization of the CB1R antagonist, without inducing a CNS effect.

In some configurations of any of the nanocarriers according to the invention, the CB1R antagonist is a central nervous system (CNS) active CB1R antagonist.

In some configurations of any of the nanocarriers according to the invention, the CNS effects associated with administration of the CNS acting CB1R antagonist are diminished or substantially reduced.

In some configurations of any of the nanocarriers according to the invention, the side effects are psychiatric side effects.

In some configurations of any of the nanocarriers according to the invention, the nanocarriers have a size between 80 and 500 nm.

In some configurations of any of the nanocarriers according to the invention, the nanocarriers have a size between 100 and 300 nm.

In some configurations of any of the nanocarriers according to the invention, the polymer is a biodegradable polymer having a neutral or a negative surface charge.

In some configurations of any of the nanocarriers according to the invention, the nanocarrier having a zeta potential between zero and minus 10.

In some configurations of any of the nanocarriers according to the invention, the nanocarriers are formed of a biodegradable hydrophobic polymer.

A peripherally restricted nanocarrier comprising a central nervous system (CNS) acting CB1R antagonist, for selective modulation of a peripheral CB1R, wherein the nanocarrier is PLGA nanocarrier having a size between 100 and 300 nm and a zeta potential between zero and −10 (minus 10) mV, and wherein the PLGA is an acid-terminated or an ester terminated PLGA having a molecular weight between 30 and 100 kDa.

In some configurations of any of the nanocarriers according to the invention, the nanocarriers are of a PLGA is of a molecular weight of between 50 and 100 kDa.

In some configurations of any of the nanocarriers according to the invention, the molecular weight of the polymer, e.g., PLGA, is 50 or 70 kDa.

In some configurations of any of the nanocarriers according to the invention, the CB1R antagonist is a neutral antagonist or an inverse agonist of the CB1R receptor having an unencapsulated form capable of crossing the blood-brain barrier and causing CNS mediated side effects.

In some configurations of any of the nanocarriers according to the invention, the antagonist is an agent capable of effective modulation of a metabolic disease or pathology.

In some configurations of any of the nanocarriers according to the invention, the metabolic diseases is NAFLD.

In some configurations of any of the nanocarriers according to the invention, the antagonist is an agent capable of effective modulation of a metabolic disease selected from obesity-induced dyslipidemia, hepatic steatosis, liver injury, insulin resistance, reversion of liver weight, elevated hepatic triglyceride content, hepatocyte ballooning, fat accumulation, hepatocellular damage, improvement of insulin sensitivity, reduction of hyperinsulinemia, chronic kidney disease, diabetes, hypertension, and improvement of Homeostatic Model Assessment for Insulin Resistance (HOMA-IR).

In some configurations of any of the nanocarriers according to the invention, the CB1R antagonist is an anorectic drug.

In some configurations of any of the nanocarriers according to the invention, the antagonist is rimonabant or an analogue or isostere or a derivative thereof.

A pharmaceutical composition comprising a CB1R antagonist contained in peripherally restricted nanocarriers according to any embodiment of the invention.

In some configurations of any of the compositions according to the invention, the composition is in a form suitable for injection.

In some configurations of any of the compositions according to the invention, the composition is in a form suitable for administration via subcutaneous (sc), intramuscular (im), intravenous (iv), or intraperitoneal (ip) administration.

A method for treating a metabolically associated disease or pathology, the method comprising administering to a subject in need of such treatment a therapeutically effective amount of a CB1R antagonist contained in peripherally restricted nanocarriers, according to any embodiment of the invention, wherein the treatment does not induce CNS derived side effects.

A method of improving a condition of a subject suffering from a metabolically-associated disease or pathology, without inducing CNS induced side effects, the method comprising administering to said subject a composition comprising nanocarriers containing a CB1R antagonist according to any embodiment of the invention.

A method of peripheralization of a CNS-active CB1R antagonist without inducing CNS-derived side effects, the method comprising containment of said CNS-active CB1R antagonist in nanocarriers and administering said nanocarriers to the subject.

A method of treating or preventing a non-alcoholic fatty liver disease (NAFLD) or type 2 diabetes (T2D) or dylipidemia in a subject, the method comprising administering to said subject a composition comprising nanocarriers comprising a CB1R antagonist suitable for treatment or prevention of the NAFLD or T2D, wherein said nanocarrier is configured for peripheralization of the CNS acting CB1R antagonist, without inducing a CNS effect.

In some configurations of any of the methods according to the invention, the CB1R antagonist is a CNS active CB1R antagonist.

In some configurations of any of the methods according to the invention, the metabolically-associated disease or pathology is a non-alcoholic fatty liver disease (NAFLD).

In some configurations of any of the methods according to the invention, the NAFLD comprises non-alcoholic fatty liver, non-alcoholic steatohepatitis (NASH), cirrhosis, and liver cancer.

In some configurations of any of the methods according to the invention, the metabolically-associated disease or pathology is one or more of metabolic syndrome, obesity-induced dyslipidemia, hepatic steatosis, liver injury, insulin resistance, reversion of liver weight, elevated hepatic triglyceride content, hepatocyte ballooning, fat accumulation, hepatocellular damage, improvement of insulin sensitivity, reduction of hyperinsulinemia, and improvement of Homeostatic Model Assessment for Insulin Resistance (HOMA-IR).

In some configurations of any of the methods according to the invention, the disease is dyslipidemia, hepatic steatosis, fatty liver, type-2 diabetes, insulin resistance, chronic kidney disease, and obesity.

In some configurations of any of the methods according to the invention, the disease is obesity.

In some configurations of any of the methods according to the invention, the metabolically-associated disease or pathology is manifested by an increase in liver weight, elevated hepatic triglyceride content, hepatocyte ballooning, fat accumulation, hepatocellular damage, decreased insulin sensitivity, hyperinsulinemia, reduced body weight, ameliorated type-2 diabetes, or prevented chronic kidney disease.

A pharmaceutical comprising a plurality of nanocarriers, each containing at least one CB1R antagonist and/or a CNS-active CB1R antagonist, wherein the pharmaceutical composition is an injectable composition suitable for administration to a subject, wherein the nanocarriers are of a size, surface charge and composition selected to prevent the nanocarriers from crossing the brain blood barrier of the subject.

In some configurations of any of the compositions according to the invention, the nanocarriers are characterized by one or more of:
a. the nanocarriers are of an acid- or ester-terminated PLGA;
b. the nanocarriers are of a PLGA having a molecular weight between 30 and 100 kDa;
c. the nanocarrier are of a size between 80 and 100 nm; and
d. the nanocarriers have surface zeta potentials between zero and −10.

In some configurations of any of the nanocarriers according to the invention, the CB1R antagonist is a CNS active CB1R antagonist capable of induing CNS derived side effects when provided in a form other than the nanocarrier.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D depict the physicochemical properties of Rimo-NPs. (A) Formulation. (B) Transmission electron microscopy (TEM) micrographs of Rimo-NPs immediately after lyophilization (Uranyl acetate negative staining, magnification 25 K, scale bar=500 nm). (C) Long-term stability (size and content) of lyophilized Rimo-NPs stored at −20° C. for 2 years. (D) Stability of Rimo-NPs in terms of size following incubation in 10% FBS solution in comparison to PBS solution. Data represents the mean±SEM from 3 independent formulations per condition.

FIGS. 2A-C depict stability assessment of Rimo-NPs. (A) The leakage of rimonabant from NPs stored at 4° C. in 50% human plasma in PBS, before and after washing Rimo-NPs with 10% albumin solution, upper line (circles) and the middle line (squares), respectively, and after washing and reconstituting the lyophilized powder, the lower line (triangles). (B) The effect of temperature on the release kinetics of rimonabant from washed and reconstituted Rimo-NPs. (C) Transmission electron microscopy (TEM) micrographs of Rimo-NPs depicting the unaffected spherical shape of the lyophilized NPs under storage (Uranyl acetate negative staining, magnification 25 K, scale bar=500 nm). Data represents the mean±SEM from 3 independent formulations per condition.

FIG. 3 depicts DSC thermogram of rimonabant base, rimonabant HCl, Blank-NPs, and Rimo-NPs.

FIGS. 4A-C depict Rimo-NP biodistribution in mice. (A) Rimo-NPs (Cy5-PLGA labeled) were administered ip to C57Bl/6JHsd mice, and examined 1, 4, 8, and 24 h after injection. Representative organ micrographs harvested 1 h after treatment (left column) and scanning micrographs by means of a Typhoon scanner (right columns). (B) The quantified fluorescence intensities in each organ are normalized to unlabeled NP-treated animals. (C) The accumulation of rimonabant in organs was also evaluated by analyzing the rimonabant levels in the liver, kidney, spleen, fat, lung, and brain 1 h post-injection (1 mg/kg, ip or iv). Data represents the mean±SEM of 3 mice per group. * p<0.05 relative to free rimonabant levels in the same tissue and the route of administration.

FIG. 5 presents rimonabant levels in the liver, kidney, fat, lung, spleen, and brain 1 h after of iv administration of free rimonabant and Rimo-NPs at 0.01-3.0 mg/kg. Data represents the mean±SEM from 3 mice per group. * p<0.05 relative to free rimonabant levels at the respective dose.

FIGS. 6A-B presents Rimonabant levels in the liver 4- and 24-hours following iv (A) or ip (B) administration of free rimonabant (Rimo-free) and Rimo-NPs at a dose of 1 mg/kg. Data are presents as mean±SEM of 3 mice per group. * p<0.05 relative to levels at the respective time after administration of free rimonabant.

FIGS. 7A-E provide Rimo-NP uptake by liver cells. (A) FACS gating strategy demonstrating the increased accumulation of Rimo-NPs in the primary hepatocytes containing the parenchymal fraction (CD45-negative cells) as well as in NPCs (CD45-positive cells). (B) Representative images of Rimo-NP (Cy5-PLGA labeled) uptake by liver hepatocytes (arrows) in mice 1 h post-treatment (ip; 140 mg/kg NPs, 200 μL). (C) Representative image of Rimo-NP (Cy5-PLGA labeled) uptake by Kupffer cells (arrows) or non-Kupffer cells (arrowheads) in mice 1 h post-treatment (ip; 140 mg/kg NPs, 200 μL). For B and C, representative confocal microscopy images are shown, the co-localization of the NPs in hepatocytes or Kupffer cells is shown in the merged images. Magnification x60, scale bar=20 μm. (D) Qualitative and (E) quantitative assessments of Rimo-NP (Cy5-PLGA labeled) uptake by primary mouse hepatocytes in culture. The fluorescence intensities are normalized to cells treated with unlabeled NPs; magnification x60, scale bar=25 μm. Data represent the mean±SEM of five independent experiments, *p<0.05.

FIG. 8 shows toxicity of Rimo-NPs. Cell viability was measured by incubating primary hepatocytes with free rimonabant, empty NPs, and Rimo-NPs for 24-48 h. Cell viability was normalized to vehicle-treated cells. Data represents the mean±SEM from three independent experiments. Polyethyleneimine (PEI) was used as a positive

FIGS. 9A-F depict Rimo-NPs do not induce centrally mediated side effects. (A) Free rimonabant, but not Rimo-NPs increased the ambulatory activity both after ip and iv administration of 1 mg/kg. Data represents the mean±SEM of 4 mice per group. * p<0.05 relative to vehicle (Veh; free PLGA-NPs), *p<0.05 relative to free rimonabant. (B) Free rimonabant at 1 and 10 mg/kg ip, but not Rimo-NPs at 1 mg/kg ip, inhibited WIN-55,212 (3 mg/kg, ip)-induced catalepsy as measured by the bar assay. Data represent the mean±SEM from 7-26 mice per group. * p<0.05 relative to the corresponding vehicle (4% DMSO, 1% Tween80, 95% saline) w/o WIN-55,212, ^{#}p<0.05 relative to the corresponding vehicle with WIN-55,212. (C, D) Free rimonabant at 10 mg/kg ip, but not Rimo-NPs or free rimonabant, both at 1 mg/kg ip, induced an anxiogenic effect in the EPM. Data represents the mean±SEM from 10-24 mice per group. * p<0.05 relative to the corresponding vehicle (4% DMSO, 1% Tween80, 95% saline), #p<0.05 relative to free rimonabant at 10 mg/kg. (E, F) Free rimonabant at 10 mg/kg ip, but not Rimo-NPs or free rimonabant at 1 mg/kg ip, inhibited acute food and water intakes. Data represents the mean±SEM from 4 mice per group. * p<0.05 relative to the vehicle (Veh; free PLGA-NPs), ^{#}p<0.05 relative to free rimonabant at 10 mg/kg control. * p<0.05 relative to vehicle-treated cells.

FIGS. 10A-B show the inability of Rimo-NPs to induce CNS-mediated hyperactivity. Free rimonabant, but not Rimo-NPs increased the ambulatory activity after both iv (A; 0.1, 1, and 3 mg/kg) and ip (B; 1 and 3 mg/kg) administrations. Hyperactivity was measured by counting the total distance (in meters) the mice traveled in the cage for 4 hours post-injection. Data represents the mean±SEM from 4 mice per group. * p<0.05 relative to vehicle (Veh; free PLGA-NPs), #p<0.05 relative to free rimonabant.

FIGS. 11A-K depict the metabolic effects of chronic treatment with free rimonabant or Rimo-NPs in DIO mice. Mice on STD or HFD for 14 weeks were treated with vehicle (Veh; free PLGA-NPs) or 1 mg/kg/d, ip, of free rimonabant or Rimo-NPs for 28 days. Free rimonabant significantly reduced body weight (A) and fat mass (B), increased lean mass (C), without affecting the food intake (D). Serum leptin levels were significantly reduced by free rimonabant and to a lesser extent by Rimo-NPs (E). Indirect calorimetry assessment over a 12 h period in the dark period revealed that free rimonabant but not Rimo-NPs resulted in an upregulation of oxygen consumption (VO₂; F), total energy expenditure (TEE; G), and fat oxidation (FO; H) without affecting the ambulatory activity (I), voluntary activity (J), and the total distance (K). Data represent the mean±SEM from 7 mice per group, *p<0.05 relative to STD-Veh, ^{#}p<0.05 relative to HFD-Veh, ^{S}p<0.05 relative to HFD-free rimonabant.

FIGS. 12A-J depict weight-independent effects of Rimo-NPs in ameliorating obesity-induced dyslipidemia and hepatic steatosis. Mice on STD or HFD for 14 weeks were treated with vehicle (Veh; free PLGA-NPs) or 1 mg/kg/d, ip, of free rimonabant or Rimo-NPs for 28 days. (A) Serum triglycerides. (B) Serum total cholesterol. (C) The HDL-to-LDL ratio. Both free rimonabant and Rimo-NPs significantly reversed the HFD-induced hepatic steatosis, as measured by reductions in liver weight (D), liver-to-body weight ratio (E), hepatic triglyceride content (F), and circulating ALT levels (G). Fat deposition assessed by H&E (I) and Oil Red O (J) staining and quantification (H). Scale bar=20 or 100 μm for H&E staining and 20 or 50 μm for Oil Red-O staining, as indicated on the images. Data represent the mean±SEM from 7 mice per group, *p<0.05 relative to STD-Veh, #p<0.05 relative to HFD-Veh, $p<0.05 relative to HFD-free rimonabant.

FIGS. 13A-B show Rimo-NPs enhances hepatic fatty acid utilization/oxidation. (A) Rimo-NPs affected the gene expression profile of hepatocytes exposed to lipotoxic conditions. Primary mouse hepatocytes were exposed to 0.5 mM mixture of oleate and palmitate (O:P 2:1, respectively) in the absence/presence of Rimo-free or Rimo-NPs (1 μM, each) for 24 h. Then, mRNA was extracted from the cells and qPCR was performed to assess the expression of several genes associated with fatty-acid β-oxidation. * p<0.05 relative to the corresponding vehicle (Veh)-treated cells. (B) Mice on STD or HFD for 14 weeks were treated ip with vehicle (Veh; free PLGA-NPs) or 1 mg/kg/d free rimonabant (Rimo-free) or Rimo-NPs for 28 days. Changes in mRNA levels of fatty acid β-oxidation genes. Data are presented as mean±SEM of 7 mice per group, *p<0.05 relative to STD-Veh, ^{#}p<0.05 relative to HFD-Veh.

FIG. 14 shows Rimo-NP biodistribution in diet-induced obese mice. The accumulation of rimonabant in organs was evaluated by analyzing the rimonabant levels in the liver, kidney, spleen, fat, lung, brain, and serum 18 h post chronic 28-day injection (1 mg/kg, ip) to high-fat diet-induced obese mice. Data represents the mean±SEM of 3 mice per group. * p<0.05 relative to free rimonabant levels in the same tissue.

FIGS. 15A-H show weight-independent effects of Rimo-NPs in ameliorating obesity-induced insulin resistance. Mice on STD or HFD for 14 weeks were treated with vehicle (Veh; free PLGA-NPs) or 1 mg/kg/d, ip, of free rimonabant or Rimo-NPs for 28 days. Free rimonabant, but not Rimo-NPs, reversed obesity-induced glucose intolerance (A-C). Both Rimo-NPs and free rimonabant improved insulin sensitivity (D-E), reduced hyperinsulinemia (F), HOMA-IR (G), and ISI (H). Data represent the mean±SEM from 7 mice per group, *p<0.05 relative to STD-Veh, #p<0.05 relative to HFD-Veh, ^{S}p<0.05 relative to HFD-free rimonabant.

FIG. 16 shows water consumption-to-urine secretion ratio following chronic treatment with Rimo-NPs. Mice on a standard diet (STD) or high-fat diet (HFD) for 14 weeks were treated with vehicle (Veh; free PLGA-NPs) or 1 mg/kg/d, ip, of free rimonabant or Rimo-NPs for 28 days. Rimo-NPs reduced the HFD-induced elevation in the water consumption-to-urine secretion ratio. Data represents the mean±SEM from 7 mice per group. * p<0.05 relative to STD-Veh, #p<0.05 relative to HFD-Veh.

FIGS. 17A-B provdie Cryo-TEM images of the initial formulation (A; 5 mg rimonabant, 95 mg 16 kDa PLGA) and one of the final optimized formulations (B; 15 mg rimonabant, 65 mg 70 kDa PLGA, 20 mg OCA, and 30 mg MCT oil).

FIGS. 18A-B demonstrate that similar brain (A) and circulating (B) levels of rimonabant injected (at a dose of 10 mg/kg, iv) either as a free solution or encapsulated in different NP formulations, as described in Table 3. Data presented represent the mean±SEM of 3-8 animals per group.

FIGS. 19A-D depict release of rimonabant base from PLGA 16 kDa acid-terminated NPs, prepared using a single-(A, B) or double-(C, D) emulsion evaporation technique. The release test was performed in 50% human serum at 4° C. Measurements were taken before (A, C) and after (B, D) the preparation was washed with a 10% albumin solution. Data are presented as the mean±SEM of 2 samples at each time point.

FIGS. 20A-D depict release of rimonabant-base from 100 kDa ester-terminated PLGA NPs prepared using a single-(A, B) or double-(C, D) emulsion evaporation technique. The release test was performed in 50% human serum at 4° C. Measurements were made before (A, C) and after (B, D) the NP preparation was washed with a 10% albumin solution. Data are presented as the value of 1 sample at each time point.

FIGS. 21A-D depict release of rimonabant-HCl from 100 kDa ester-terminated PLGA NPs prepared using a single-(A, B) or double-(C, D) emulsion evaporation technique. The release test was performed in 50% human serum at 4° C. Measurements were made both before (A, C) and after (B, D) the preparation was washed with a 10% albumin solution. Data are presented as values of 1 sample at each time point.

FIGS. 22A-D depict release test of rimonabant-base from 50 kDa acid-terminated PLGA NPs prepared using the single-(A, B) or double-(C, D) emulsion evaporation technique. The release test was performed in 50% human serum at 4° C. Measurements were made before (A, C) and after (B, D) the preparation was washed with a 10% albumin solution. Data are presented as the mean±SEM of 1-2 samples at each time point.

FIGS. 23A-D depict release of rimonabant-HCl from 50 kDa acid-terminated PLGA NPs prepared using single-(A, B) or double-(C, D) emulsion evaporation techniques. The release test was performed in 50% human serum at 4° C. Measurements were made both before (A, C) and after (B, D) the preparation was washed with a 10% albumin solution. Data are presented as the mean±SEM of 1-3 samples at each time point.

FIGS. 24A-B show cryo-TEM images of the liposomes. Unilamellar liposome formulation (A), and multilamellar liposome formulation (B).

FIGS. 25A-B depict brain and circulating levels of rimonabant (liposomes). Similar brain (A) and circulating (B) levels of rimonabant injected (at a dose of 3 mg/kg, iv) either as a free solution or encapsulated in different liposome formulations as described in Table 10. Data represent the mean±SEM of 3 animals per group.

### DETAILED DESCRIPTION OF EMBODIMENTS

### Materials and Methods

### Reagents.

Rimonabant hydrochloride was purchased from Glentham (Cat #GP8358). PLGA, which has a lactide-glycolide ratio of 50:50 and a MW of 30,000-60,000 (acid-terminated), was purchased from Lactel Absorbable Polymers (Cat #B6013-2). PLGA-Cyanine 5 endcap, which has a lactide-glycolide ratio of 50:50 and a MW of 30,000-55,000 (acid-terminated), was purchased from PolySciTech (Cat #AV034). Poly(vinyl alcohol) (PVA, 30-70 kDa) was purchased from Sigma-Aldrich (Cat #P8136). Phosphate salts were purchased from Biological Industries (Cat #11-223-1G). AM251 was purchased from Cayman Chemical Company (Cat #71670).

### Nanoparticles Preparation.

Various formulation attempts have been experimented. The selected Rimo-NP formulation was prepared by the emulsion evaporation technique. Briefly, a solution of 10 mg of rimonabant in 200 μL EtOH was added to 3 mL of dichloromethane (DCM) containing 3% PLGA. The solution was added dropwise to a 2% PVA solution (in 25 mL 1×PBS buffer); then, it was sonicated for 105 sec at 80% amplitude over an ice bath to form a single emulsion (O/W). DCM was eliminated by evaporation under reduced pressure using a rotary evaporator (Buchi, Switzerland), which resulted in NP formation. The NPs were recovered by ultracentrifugation (20,000 rpm for 20 min at 4° C.), then washed twice (with a 10% albumin solution, and finally with 1×PBS) to remove PVA and unentrapped rimonabant. Next, the pellet was resuspended in a 10% sucrose solution and lyophilized. Dry lyophilized NPs were stored at −20° C. until use. Drug-free NPs (empty NPs) were prepared by the same procedure, except that rimonabant was omitted. Fluorescent NPs were prepared by replacing 10% of the PLGA with Cyanine-5-labeled PLGA.

### Physicochemical Characterization of NPs.

### Size, Surface Charge, and Morphology

NP size and surface charge (Z potential) were determined by dynamic light scattering (DLS) at room temperature (Zetasizer Nano-ZSP, Malvern Instruments, UK). An aliquot (50 μL) of the NP suspension, obtained before lyophilization or after resuspending the lyophilized NPs in 1×PBS buffer, was added to 1 mL HPLC water. NP morphology was assessed utilizing transmission electron microscopy (TEM, JEM-1400 Plus, JEOL Ltd., Tokyo, Japan). Briefly, 5 μL samples were stained with NanoVan™ (Nanoprobes, NY, USA) and then placed on formvar/carbon-coated copper 200 mesh grids (EMS), and mixed with 5 μL of NV for 5-10 sec. Excess stain was blotted off and grids were dried. The grids were viewed with Jeol® JEM-1400 Plus TEM (Jeol®, Tokyo, Japan). The grids were equipped with an ORIUS SC600 CCD camera (Gatan®, Abingdon, United Kingdom); the Gatan Microscopy Suite program was used (DigitalMicrograph, Gatan®, UK).

### Rimonabant Loading Efficiency

For each batch, 1-2 mg of NPs was dissolved in 0.1 mL of HPLC water and 0.9 mL of acetonitrile. Samples were centrifuged at 13,000 rpm for 10 min at room temperature; then, the supernatant was analyzed by HPLC (Dionex UltiMate 3000, Thermo Scientific); the equipment consisted of an UltiMate 3000 SD pump and a UV-Vis Absorbance (DAD, MWD, VWD). The separation was performed on a Phenomenex C18 column (4.6 mm×150 mm) at 225 nm. Next, the mobile phase was prepared with acetonitrile and water (90:10), and was pumped at a rate of 0.6 mL/min at 25° C. The injection volume for analysis was 10 μL. The concentration of rimonabant was calculated against an appropriate calibration curve. Rimonabant's loading and encapsulation efficiency (%) were calculated using the following equations:

$$Rimonabant ' s loading ( \% ) = \frac{Rimonabant weight {( mg )}^{*} \times 100}{NP weight ( mg )}$$

$$Encapsulation efficiency ( \% ) = \frac{Actual rimonabant loading ( mg / mg ) \times 100}{Theoretical rimonabant loading {( mg / mg )}^{**}}$$

*Calculated based on the spectrophotometric assay.
**Calculated based on the initial amount of rimonabant used for NP preparation.

### Rimonabant-Loaded NP Stability

To examine the stability of the formulation, which was stored at −20° C., the size and content of lyophilized NPs were examined at different time points over a period of 2 years by DLS and HPLC.

### In Vitro Drug Release

The drug release from rimonabant-loaded NPs was carried out by ultracentrifugation of the NPs and by measuring the amount of rimonabant in the supernatant. Next, 0.1 mL of rimonabant-loaded NPs was added into 0.9 mL of 50% human serum in 1×PBS at 4° C. and 37° C. Samples were collected at specific time points, centrifuged at 45,000 rpm for 75 min at 4° C.; then the drug content in the supernatant and residue was measured using an HPLC.

### Cell Cultures.

Primary mouse hepatocytes were isolated. Cells were cultured in William's E complete medium (Rhenium, Israel), 2 mM L-glutamine, 100 units/mL penicillin, and 100 mg/mL streptomycin at 37° C. and in a humidified 5% CO₂/95% air atmosphere.

### In Vitro Cytotoxicity.

The cytotoxicity of the rimonabant-loaded NPs was examined in comparison with empty NPs and a free drug solution. Primary hepatocytes were seeded in 12-well plates (200,000 cells/well) containing the complete growth medium. The cells were left to properly adhere for 1 day, and then incubated with different solutions for 24 and 48 h. Cell viability was determined by XTT assay PMS (N-methyl dibenzopyrazine methyl sulfate). The plates were quantified by a plate reader at a wavelength of 450-630 nm. Cell viability was normalized to vehicle-treated cells.

### Cellular Uptake of the NPs.

Primary hepatocytes were seeded on coverslips in 12-well plates (200,000 cells/well) containing a complete growth medium and were left overnight to adhere. Cells were incubated for 1, 3, 6, and 24 h with 4.4 mg/mL fluorescent NPs (Cy5-PLGA NPs). The nucleus was stained with DAPI. Next, the cells were washed three times with 1×PBS, fixed using 4% formaldehyde solution for 10 min, washed again with 1×PBS, and mounted onto a microscope slide. Slides were analyzed using a Nikon A1R Confocal laser scanning microscope (magnification of ×60). Cells treated with unlabeled NPs were used as controls.

### Animals and the Experimental Protocol.

The experimental protocol used was approved by the Institutional Animal Care and Use Committee of the Hebrew University, which is an AAALAC International accredited institute. To generate diet-induced obesity (DIO; body weight>42 g), six-week-old male C57B1/6JHsd mice (Envigo, Israel) were fed either a high-fat diet (HFD; 60% of calories from fat, 20% from protein, and 20% from carbohydrates; Research Diet, D12492), or a standard diet (STD; 14% fat, 24% protein, 62% carbohydrates; NIH-31 rodent diet) for 16 weeks. To assess the metabolic effect of the formulations, the mice were treated ip with Rimo-NPs, a free solution of rimonabant (1 mg/kg, each), or empty NPs (vehicle) for 4 weeks. Body-weight and food intake were measured daily. Total body fat and lean masses were determined by EchoMRI100H™ (Echo Medical Systems LLC, Houston, TX, USA). At 24 h, urine was collected one week before euthanasia using mouse metabolic cages (CCS2000 Chiller System, Hatteras Instruments, NC, USA). Mice were euthanized by cervical dislocation under anesthesia, and the kidneys, brain, liver, fat pads, spleen, lungs, and heart were removed and weighed, and samples were either snap-frozen or fixed in buffered 4% formalin. Trunk blood was collected to determine the biochemical parameters.

### Blood and Liver Biochemistry.

Serum levels of cholesterol, triglycerides, high-density lipoprotein (HDL), low-density lipoprotein (LDL), and alanine aminotransferase (ALT) were determined by using the Cobas C-111 chemistry analyzer (Roche, Switzerland). Serum insulin and leptin levels were measured by ELISAs (Merck, Cat #EZRMI-13K and #E6082-K, respectively). Fasting blood glucose was measured using a glucometer. Liver tissues were extracted as known, and their triglyceride content was determined using the Cobas C-111 chemistry analyzer (Roche, Switzerland).

Glucose Tolerance (ipGTT) and Insulin Sensitivity Tests (ipIST).

Mice that underwent overnight fasting were injected with glucose (1.5 g/kg, ip), followed by a tail blood collection at 0, 15, 30, 45, 60, 90, and 120 minutes. The blood glucose levels were determined using a glucometer. The following day, the mice underwent fasting for 6 h before receiving insulin (0.75 U/kg, ip; Eli Lilly); their blood glucose levels were determined at the same intervals as above. The homeostasis model assessment insulin resistance (HOMA-IR) was calculated as ([μU/mL] X fasting plasma glucose [mmol/L]/22.5). The insulin sensitivity index (ISI) was calculated as 1/(glucose×insulin)×1000, with glucose expressed as mg/dL and insulin as mU/L.

### Histopathological Analyses.

Five μm paraffin-embedded liver sections from 7 animals per group were stained with H&E staining. Liver images were captured with a Zeiss AxioCam ICc5 color camera mounted on a Zeiss Axio Scope.A1 light microscope and taken from 10 random 40× fields of each animal. Lipid staining was conducted using an Oil Red O staining kit (Cat #ab150678; Abcam) according to the manufacturer's protocol. Briefly, at a 10 mm optimal cutting temperature (O.C.T) (Cat #4583; SciGen, Inc., Gardena, CA, USA), compound-embedded liver sections were placed in propylene glycerol, followed by an Oil Red O solution and hematoxylin staining. Stained sections were photographed as mentioned above and positive red areas were quantified in a blinded manner using Image J software with a minimum of 10 random liver sections per mouse.

### Biodistribution of Rimonabant-Loaded NPs.

The biodistribution of Rimo-NPs, in comparison with free rimonabant, was determined in two cohorts of mice: The first cohort included male 10-11-week-old C57b1/6JHsd mice (Envigo, Israel) injected with Rimo-NPs or free rimonabant (iv: 0.01, 0.1, 1, 3 mg/kg; ip: 1 mg/kg). The NPs were suspended in 1×PBS (15-450 μg/mL), and the free drug was dissolved in 4% DMSO, 1% Tween 80, and saline (0.1-3.0 mg/mL). The mice were euthanized 1-, 4-, and 24-h post-injection. The second cohort included HFD-fed obese mice treated chronically (28 days) with Rimo-NPs or free rimonabant (1 mg/kg, ip). The mice were euthanized 18 h following the last injection. Blood was collected, and mice were perfused with 1×PBS (via the left ventricle until the effluent from the right atrium was clear of blood), and the brain, lungs, liver, kidneys, fat, and spleen were harvested.

Tissues and serum samples were processed as follows: Total proteins were precipitated with ice-cold acetone/Tris buffer (50 mM, pH 8.0). Then, the samples were homogenized in ice-cold methanol/Tris buffer (50 mM, pH 8.0, 1:1) containing AM251 10 μg/mL as an internal standard. Homogenates were then extracted with CHCl₃:MeOH (2:1, vol/vol) and washed two times with ice-cold CHCl₃, dried under nitrogen flow, and reconstituted with MeOH. Rimonabant levels in the samples were determined by liquid chromatography/tandem mass spectrometry (LC-MS/MS).

The analyses were conducted on a TSQ Quantum Access Max triple quadrupole mass spectrometer (Thermo Scientific, San Jose, CA, USA) coupled with an UHPLC system, which included a Dionex Pump and an Accela Autosampler (Thermo Scientific, San Jose, CA, USA). A Kinetex (Phenomenex, Torrance, CA, USA) column (C18, 2.7 μm particle size, 50×2.1 mm) was used for separation. Gradient elution mobile phases consisted of 0.1% formic acid in water (phase A) and 0.1% formic acid in acetonitrile (phase B). Gradient elution (0.3 mL/min) was held at 40% B for 0.7 min, followed by a linear increase to 85% B for 6.3 min, followed by a linear increase to 95% B for 0.2 min, and it was maintained at 95% B for 3.3 min. Then, it decreased linearly to 40% B for 0.5 min, and it was maintained at 40% B for 6.0 min. Rimonabant was detected in a positive ion mode using electron spray ionization (ESI) and the multiple reaction monitoring (MRM) mode of acquisition. The mass spectrometer parameters were set as follows: spray voltage 4500 V; vaporizer temperature 370° C.; capillary temperature 260° C.; sheath and auxiliary gases 35 and 30 arbitrary units, respectively; argon was used as the collision gas.

The molecular ions and fragments for each compound were measured as follows: m/z 463/363 (quantifier), m/z 463/300 (qualifier) for rimonabant (collision energy: 29 V and 40 V, respectively), and m/z 555/454.9 (quantifier) and m/z 555/327.9 (qualifier) for AM251 (collision energy: 30 V and 49 V, respectively). TSQ Tune Software (Thermo Scientific, San Jose, CA, USA) was used to optimize the tuning parameters. Data acquisition and processing were carried out using the Xcalibur program (Thermo Scientific, San Jose, CA, USA). The amounts of rimonabant in the samples were determined against a standard curve. Values are expressed as ng/g or ng/mL in wet tissue weight or serum volume, respectively.

### Biodistribution of Cy-5 PLGA NPs.

Sixteen-week-old male C57Bl/6JHsd mice were injected ip with fluorescent NPs (PLGA covalently linked to cyanine 5). The NPs were suspended in 1×PBS (21.4 mg/mL), and 200 μL (140 mg/kg NPs) were injected per mouse. The mice were euthanized 1, 4, 8, and 24 h (n=3 in each group and 1 mouse as a control) post-injection and perfused with 1×PBS (via the left ventricle until the effluent from the right atrium was clear of blood). Finally, the brain, lungs, liver, kidneys, fat pads, and spleen were harvested. The accumulation of the fluorescent NPs in the organs was assessed by fluorescent imaging (Typhoon FLA 9500 biomolecular imager, GE Healthcare, UK), followed by image analysis (ImageJ). The mean fluorescent intensity in each organ was subtracted from that of an animal that was treated with empty NPs as a control. Immediately after the livers were scanned, they were embedded in OCT (Bar-Naor, Cat #BN62550), followed by snap freezing in dry ice; they were stored at −80° C. until cryo-sectioned by using the Leica CM1950 cryostat (Leica Biosystems, Germany).

### Immunostaining.

Liver sections were prepared as described above and frozen without a mounting medium. Following thawing at room temperature, the sections were washed three times for 5 minutes each in 1×PBS, fixed (4% formaldehyde) for 15 min, washed three times for 5 minutes each in 1×PBS, and blocked with 10% Normal Goat Serum in 1× PBS/0.05% Tween-20 for 1 h (for F4/80 staining) or blocked with 10% Normal Goat Serum, 1% BSA, 1% Triton (for albumin staining). Then, the sections were washed three times for 5 minutes each in 1×PBS. Kupffer cells were stained overnight with a rat anti-mouse F4/80 antibody (1:100 abcam, Cat #6640) and hepatocytes were stained overnight with FITC-anti mouse albumin antibody (1:10, Santa Cruz, Cat #271605) at 4° C. Slides were then washed three times for 10 minutes each in 1×PBS (for albumin) or in 1×PBS/0.05% Tween-20 (for F4/80) staining, and incubated with a goat anti-rat FITC-conjugated antibody (1:100, abcam, Cat #ab150157) for 1 h. Following three washing steps of 10 min each, the sections were embedded in Vectashield mounting media containing DAPI (abcam, Cat #ab104139), and the images were captured with a Nikon A1R Confocal laser scanning microscope (magnification of ×60).

### Flow Cytometry Analysis.

Six 10-12-week-old male C57B1/6J mice (JAX, Bar Harbour) were injected ip with fluorescent NPs (PLGA covalently linked to Cyanine 5). The NPs were suspended in 1×PBS (20.2 mg/mL), and 200 μL (140 mg/kg NPs) were injected per mouse. The mice (n=3 in each group) were euthanized 1 h post-injection; then hepatocytes and NPCs were isolated. Briefly, mice were anesthetized and perfused with the Liver Perfusion Buffer (Gibco, Cat #17701-038); then, they were washed in a digestion buffer (HBSS Biological Industries, Beit HaEmek, Cat #02-018-1A) containing 5 mM CaCl₂, 10 mM HEPES, and 25 μg/mL collagenase I & II (Liberase™, Roche Cat #5401119001). After the digestion procedure, the liver was excised and maintained in the Hepatocyte Wash Media (Gibco, Cat #17704024) on ice. Hepatocytes were released from liver lobes and then filtered through 70-μm cell strainers (SPL Life Sciences, Cat #93070). After centrifugation and washing steps, hepatocytes were re-suspended in 40% Percoll (Sigma; Cat #GE17-0891-01) in Williams E Media (Gibco, Cat #12551032), and centrifuged (60×g for 4 min) to collect live hepatocytes on the bottom of the vial. The remaining liver lobes were minced and further digested by using 2.5 mg/mL collagenase D (Roche, Cat #11088858001) and 100 ng/ml DNAse I (Roche, Cat #11284932001) at 37° C. for 30 min. After the centrifugation and washing steps, the NPC pellets were loaded on top of Percoll gradient layers (10 mL 15% Percoll on the top and 10 mL 40% Percoll on the bottom) for further centrifugation. The NPC layer in the middle was collected. Isolated hepatocytes and NPCs were first incubated with Fc-receptor blocking, anti-mouse CD16/CD32 antibody (BioLegend, #101301), then stained with FITC anti-mouse CD45 (BioLegend, #157608). Flow cytometry was performed using a CytoFLEX Flow Cytometer (Beckman Coulter, Switzerland). Unstained hepatocytes and NPCs, isolated from mice injected with unlabeled NPs, were used as negative controls.

Real-Time qPCR.

Total RNA of mouse livers or primary hepatocytes was extracted using TRIzol (Invitrogen), followed by DNase I treatment (Invitrogen), and then reverse-transcribed using an Iscript cDNA kit (Bio-Rad). Real-time PCR was conducted using iTaq Universal SYBR Green Supermix (Bio-Rad) and the CFX connect ST system (Bio-Rad). The list of mouse primers is presented in Table 1 below. The following Mus musculus genes were detected: Cpta1, Acadm, Echs1, Hadh, and Acaa2; all genes were normalized to β-actin.

TABLE 1
List of mouse primers

| | Forward primer | Reverse primer |
|---|---|---|
| Gene | (5′-3′) | (5′-3′) |
| Cpt1a | CCGTGAGGAAC | CAGGGATGCGGGAA |
| | TCAAACCTATT | GTATTG |
| Acadm | CAGCCAATGAT | CATACTCGTCACCC |
| | GTGTGCTTAC | TTCTTCTC |
| Echs1 | GGACTGTTACT | CCCACCAAGAGCAT |
| | CCAGCAAGTTC | AACCATT |
| Hadh | CCAAGAAGGGA | ACAAACTCATCTCC |
| | ATTGAGGAGAG | AGCCTTAG |
| Acaa2 | CAGAGGTGGAA | GCATGGTCTGTTTG |
| | AGCTGCTAA | CCTTTC |
| Actb | GGCTGTATTCC | CCAGTTGGTAACAA |
| | CCTCCATCG | TGCCATGT |

Cpt1a, Carnitine palmitoyltransferase 1A; Acadm, acyl-CoA dehydrogenase medium chain; Echs1, enoyl-CoA hydratase, short chain 1; Hadh, hydroxyacyl-CoA dehydrogenase; Acaa, acetyl-CoA acyltransferase; Actb, beta actin.

### Cell Cultures.

Primary mouse hepatocytes were isolated by liberase perfusion as previously described. Briefly, the liver of anesthetized mice was first perfused with calcium-free Hanks balanced salt solution supplemented with HEPES and EDTA (HBSS, Biological Industries, Beit HaEmek, Cat #02-018-1A HEPES Cat #03-025-1B), followed by perfused liberase digestion (25 μg/mL liberase in HBSS; Biological Industries, Beit HaEmek, Cat #02-015-1A with 25 mM HEPES). After digestion, the hepatocytes were released by dissociation from the lobes (centrifuged at 50×g for 2 min at 4° C.). The pellet from the first centrifugation of hepatocytes was loaded on a 90% Percoll gradient (Sigma-Aldrich; Cat #GE17-0891-01) and centrifuged at 200×g for 10 min at 4° C. The cells were then cultured on 6-well plates at a density of 4×10⁵ cells/well with planting medium (DMEM, Biological Industries, Beit HaEmek, Cat #01-050-1A) supplemented with 100 U/mL penicillin-streptomycin and 5% FBS, which, was changed after 3 h to William's E medium (Rhenium; Cat #22551022) containing 2 mM L-glutamine and 100 U/mL penicillin-streptomycin. Cells were incubated overnight at 37° C. in a humidified 5% CO₂/95% air atmosphere.

CB1R Antagonism with Free Rimonabant/Rimonabant-NPs.

Primary hepatocytes were cultured in M199 medium (Biological Industries, Beit HaEmek, Cat #01-080-1A) containing 1% fatty acid-free BSA. Free rimonabant or Rimo-NPs were added to the medium to a final concentration of 1 μM, followed by the addition of a mixed solution of sodium oleate (Sigma-Aldrich; Cat #07501) and sodium palmitate (Sigma-Aldrich; Cat #P9767) at a ratio of 2:1, to a final concentration of 0.5 mM, for 24 h. Then, the cells were harvested in Trizol for qPCR analysis.

### Multi-Parameter Metabolic Assessment.

The metabolic and activity profiles of the mice were assessed using the Promethion High-Definition Behavioral Phenotyping System (Sable Instruments, Inc., Las Vegas, NV, USA). Data acquisition and instrument control were performed using MetaScreen software version 2.2.18.0, and the obtained raw data were processed using ExpeData version 1.8.4, using an analysis script detailing all aspects of the data transformation. Mice with free access to food and water were subjected to a standard 12 h light/12 h dark cycle, which consisted of a 16 h acclimation period, followed by 24 h of sampling. Respiratory gases were measured by using the GA-3 gas analyzer (Sable Systems, Inc., Las Vegas, NV, USA), using a pull-mode, negative-pressure system. Airflow was measured and controlled by FR-8 (Sable Systems, Inc., Las Vegas, NV, USA), with a set flow rate of 2000 mL/min. Water vapor was continuously measured and its dilution effect on O₂ and CO₂ was mathematically compensated. The effective mass (eff.Mass) was calculated by ANCOVA analysis. The respiratory quotient (RQ) was calculated as the ratio between CO₂ produced to O₂ consumed, and the total energy expenditure (TEE) was calculated as VO₂×(3.815+1.232×RQ), normalized to the effective body mass, and expressed as kcal/h/kg^{eff.Mass}. Fat oxidation (FO) was calculated as FO=1.69×VO₂−1.69×VCO₂ and expressed as g/d/kg_{eff.Mass}. Ambulatory activity and position were monitored simultaneously with the collection of the calorimetry data using XYZ beam arrays with a beam spacing of 0.25 cm.

### Elevated Plus-Maze.

Anxiety-related behaviors were assessed using the EPM test. Animals were placed on the 5×5 cm central platform of an apparatus from which four arms, 30 cm×5 cm, extended. Two of the arms (the closed arms) are enclosed within 15 cm high walls, and the two other arms (the open arms) have 1 cm high rims. The whole maze is elevated 75 cm above the ground. During the 6 min test time, the number of entries to each arm type (closed or open arms and frequencies) and the time spent in each type of arm (closed or open arms and durations) during the test were measured.

### Catalepsy Test.

Catalepsy was assayed using the bar test. Briefly, mice were removed from their home cage, and their forepaws were placed on a horizontal bar, 0.5 cm in diameter, positioned 4 cm above the bench surface. Vehicle-treated mice routinely let go of the bar within 2 seconds. Cataleptic behavior was defined as the time the animals remained motionless holding onto the bar, with an arbitrary cutoff of 30 seconds. The antagonists (free rimonabant at 1 and 10 mg/kg, Rimo-NPs at 1 mg/kg) were given 30 min before the ip injection of 3 mg/kg WIN55,212. The test was performed 60 minutes after agonist administration.

### Activity Profile.

Locomotor activity was quantified by the number of disruptions of the infrared beams in 2 dimensions in the Promethion High-Definition Behavioral Phenotyping System (Sable Instruments, Inc., Las Vegas, NV, USA).

### Acute Food and Water Intake.

Precise measurements of food and water intake in male 10-12-week-old C57Bl/6JHsd mice were assessed by using the Promethion High-Definition Behavioral Phenotyping System. Briefly, mice with free access to water were fasted from food for 24 h. The mice were injected with the test drugs 30 min before they were refed. Cumulative food and water intakes were measured for 3 h.

### Statistics.

Values are expressed as the mean±SEM. An unpaired two-tailed Student's t-test was used to determine differences between the Veh- and the drug-treated groups. The results in multiple groups and time-dependent variables were compared by ANOVA, followed by a Tukey's multiple comparisons test (GraphPad Prism v6 for Windows). Significance was at p<0.05.

### Results

### Rimo-NPs Formulation and Characterization

To successfully encapsulate rimonabant in NPs or liposomes, we used various polymers (acid-or ester-terminated PLGA or PLA, having different MWs, and various lactic acid:glycolic acid ratios), diverse nano preparations (nanoprecipitation, single, or double emulsion evaporation), as well as different surfactants (Solutol or PVA). Unfortunately, neither of these methods resulted in efficient entrapment of rimonabant in the nano-drug delivery systems nor prevented its aggregation.

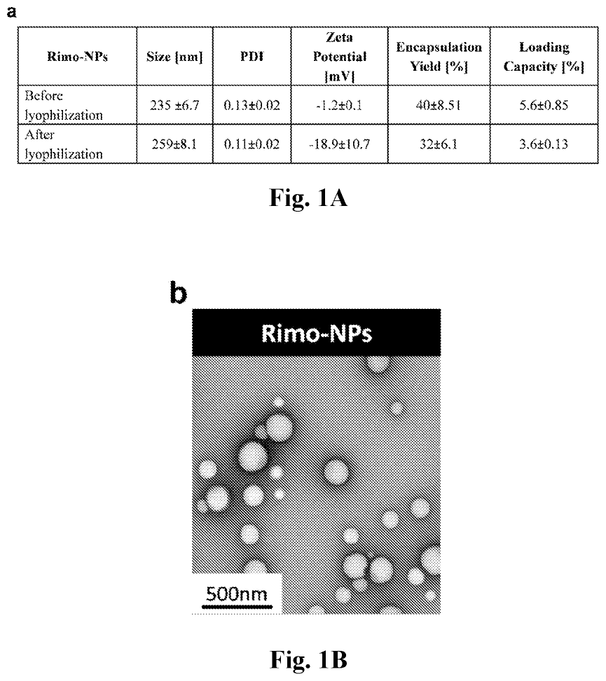

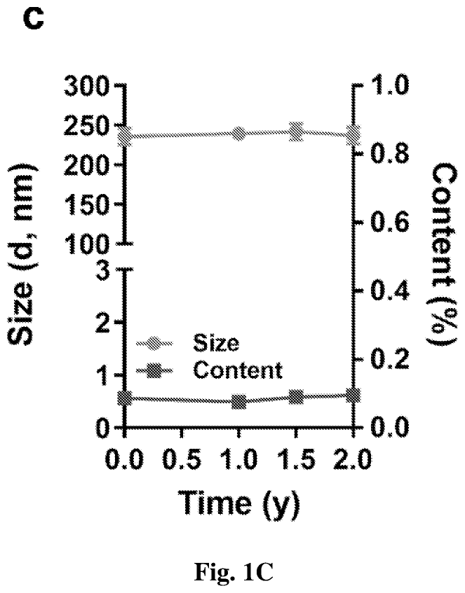

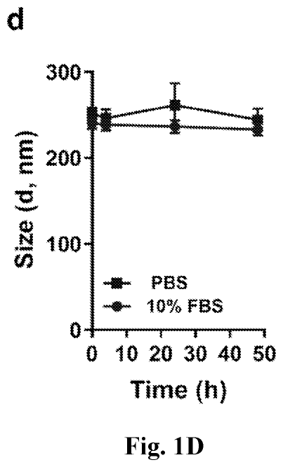

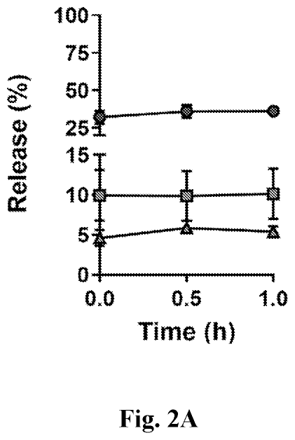

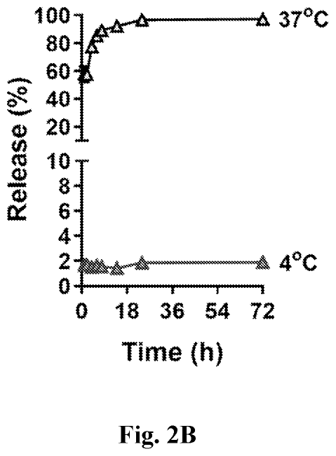

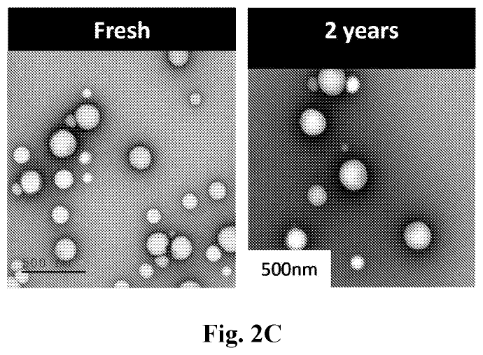

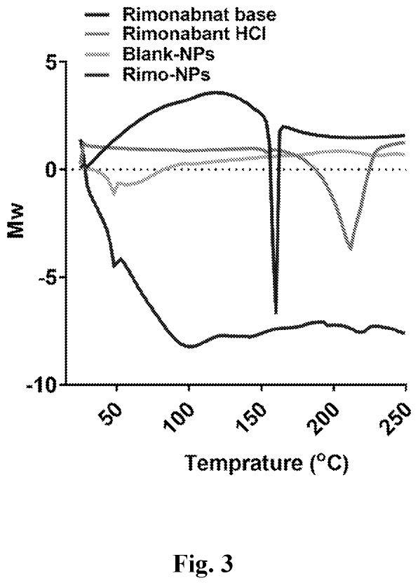

Ultimately, PLGA-based Rimo-NPs were successfully formulated and characterized with spherical geometry; they had a diameter size of ˜250 nm, a relatively narrow PDI (˜0.11±0.02), a zeta potential of −18.9±10.7 mV, a loading capacity of 3.6±0.13% rimonabant, and an encapsulation efficiency of 32.53±6.1% after lyophilization (FIGS. 1A-B). Before lyophilization, the NPs were washed with 10% albumin solution to remove loosely bound rimonabant (FIG. 2). The physicochemical properties of Rimo-NPs, in terms of size and drug concentration, were not affected by lyophilization or changed after 2 years of storage at −20° C. (FIG. 1C, and FIG. 2C). No significant protein adsorption on Rimo-NPs was detected following incubation in serum-containing media (10% FBS) in terms of size changes up to 48 h (FIG. 1d). The leakage of rimonabant from the NPs was low at 4° C.; it increased at 37° C. (FIG. 2B). Rimo in the NPs was found to be in an amorphous state, indicated by differential scanning calorimetry (DSC); it exhibited a phase transition from the crystalline, rimonabant-HCl (FIG. 3).

### Increased Peripheral Accumulation of Rimo-NPs

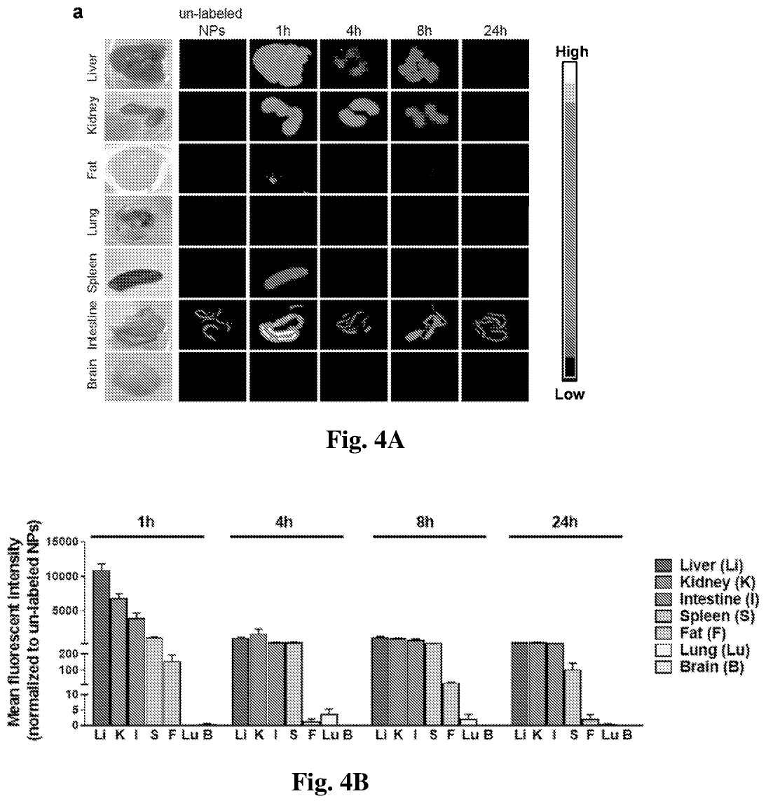

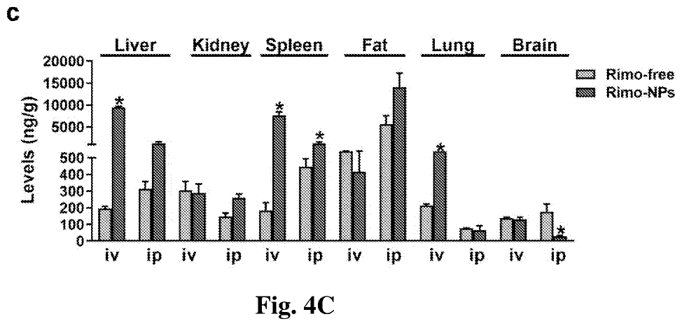

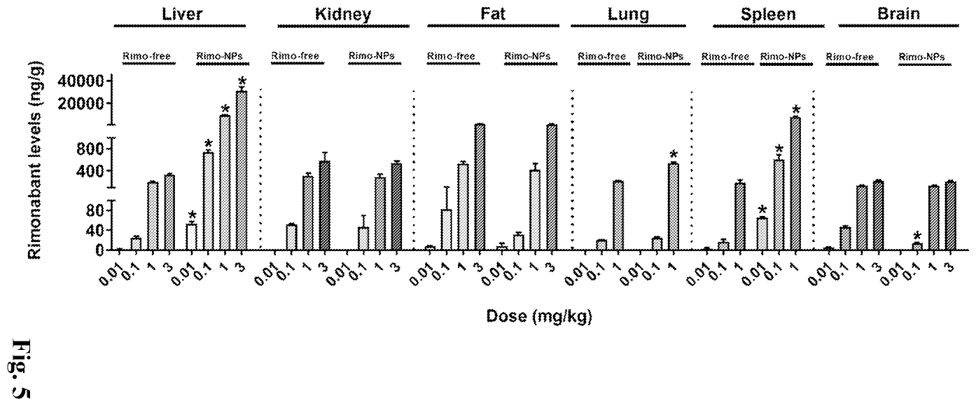

To validate our hypothesis of rimonabant peripheralization by administering the drug embedded in NPs, we carried out thorough biodistribution studies. First, we determined the fate of Cy5-labeled NPs administered intraperitoneally (ip) to mice. The NP biodistribution in the various organs was liver>kidney>intestine>spleen>fat>>>lungs; only a negligible amount was detected in the brain (FIGS. 4A-B). In contrast, rimonabant formulated in the NPs with solutol or in liposomes (Table 1) penetrated the brain. Noteworthy is the significantly higher accumulation of rimonabant in the liver and the spleen following treatment with Rimo-NPs, in comparison with animals treated with rimonabant solution in the liver and the spleen (FIG. 4C; a 4-fold and 2.6-fold higher concentration in the liver and spleen, respectively). Remarkably, the brain levels of rimonabant were significantly (5-fold) lower following Rimo-NP administration, compared with free rimonabant solution, 1 h post-ip administration (FIG. 4C). In addition, high levels of rimonabant in the liver were found following iv or ip administration of Rimo-NPs (1 mg/kg; FIG. 4C), as well as after various iv doses (0.01 to 3.0 mg/kg (FIG. 5)). The peripheralization of rimonabant, following Rimo-NP administration, was also manifested by the significantly higher levels of the drug in the lungs (1 mg/kg, iv) and in the spleen (0.01 to 1.0 mg/kg, iv), compared with free rimonabant treatments (FIG. 5).

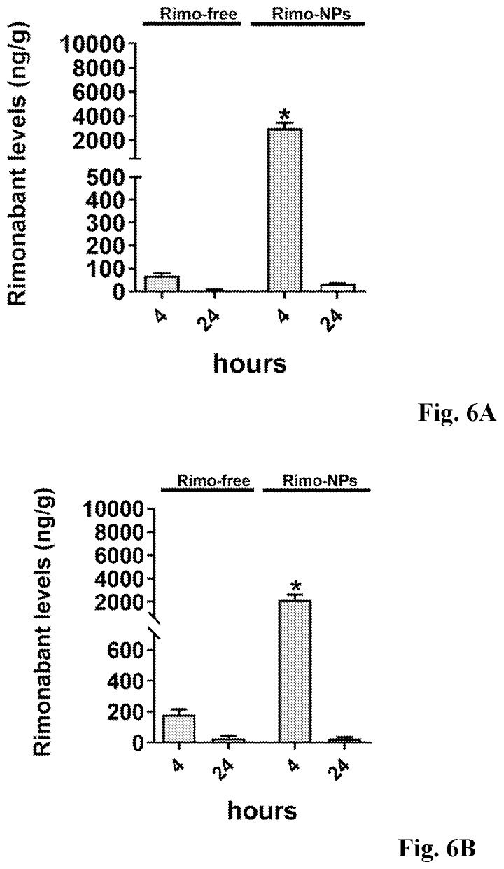

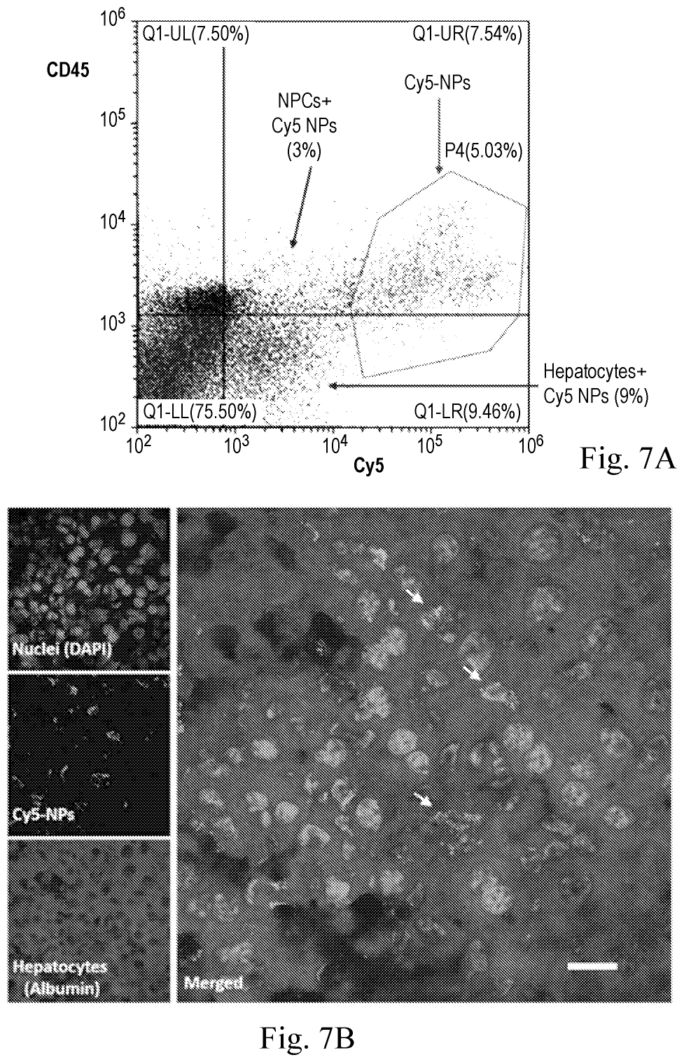

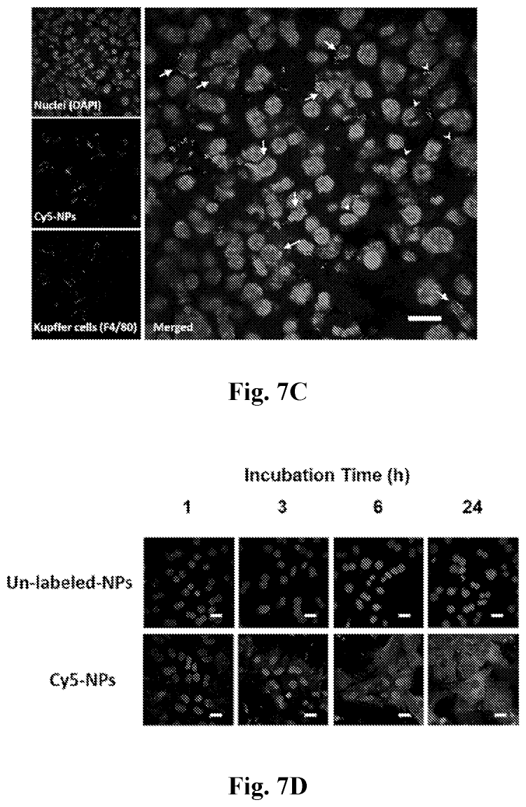

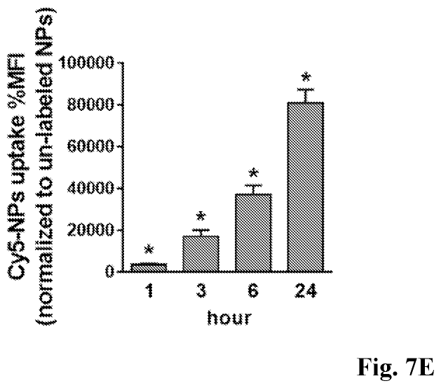

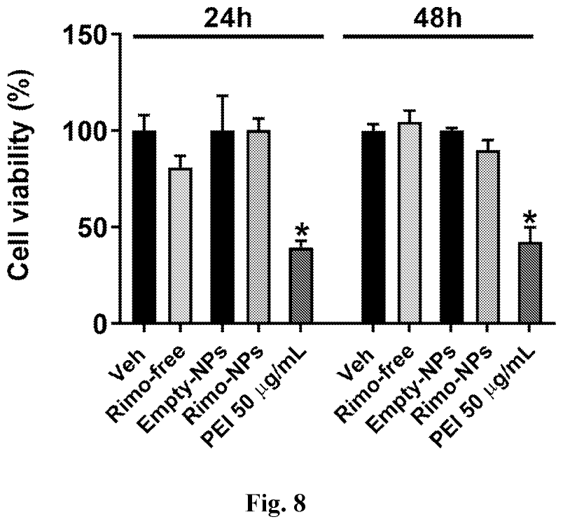

Since our targeted cells for therapy could be CB1R-expressing cells in the liver, e.g., hepatocytes or non-parenchymal cells (NPCs) such as Kupffer cells, and due to the high levels of the drug found in the liver even 4 hours after administration of Rimo-NPs (FIG. 6), we next assessed the specific cellular fate of Cy5-labeled NPs in the liver. As shown in FIG. 7A, an accumulation of Cy5-labeled NPs was found in 9% of primary hepatocytes containing a parenchymal fraction (CD45-negative cells), which were isolated from the animals 1 h after administration. The immunofluorescent analysis of liver sections, collected from mice injected with Cy5-labeled NPs, revealing that the NPs are indeed localized in hepatocytes (FIG. 7B), corroborated this finding. Additional support for Rimo-NPs' affinity to hepatocytes is the increased uptake profile of Cy5-labeled NPs detected in primary mouse hepatocytes in vitro (FIGS. 7D-E). Nevertheless, Rimo-NPs were also taken up by NPCs, since the NPs were found in a small fraction (3%) of CD45- or F4/80-positive cells, respectively (FIGS. 7A,C). Of note is that the treatment was not associated with any cellular toxicity (FIG. 8).

### Rimo-NPs do not Induce Centrally Mediated Side Effects

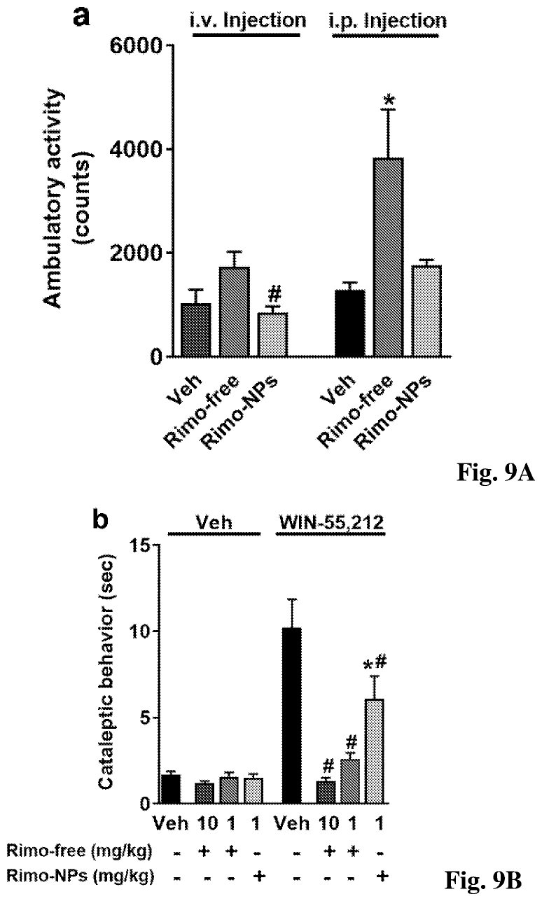

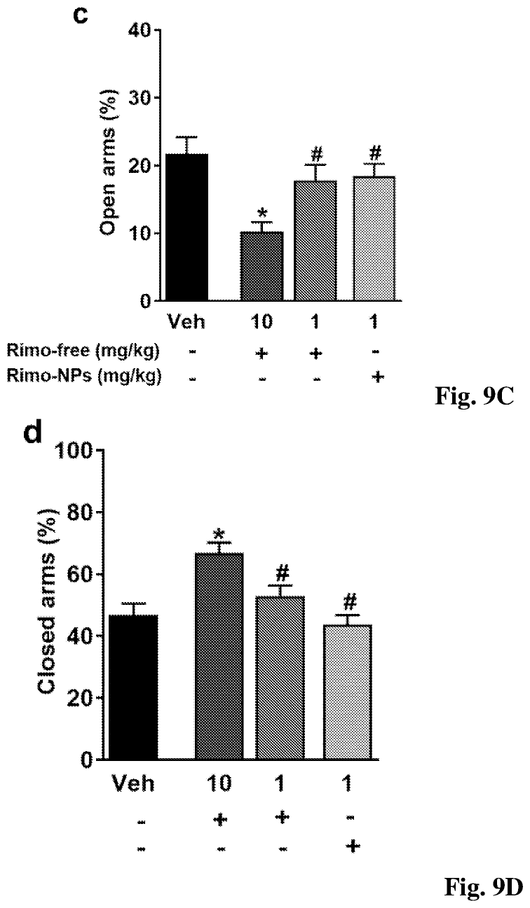

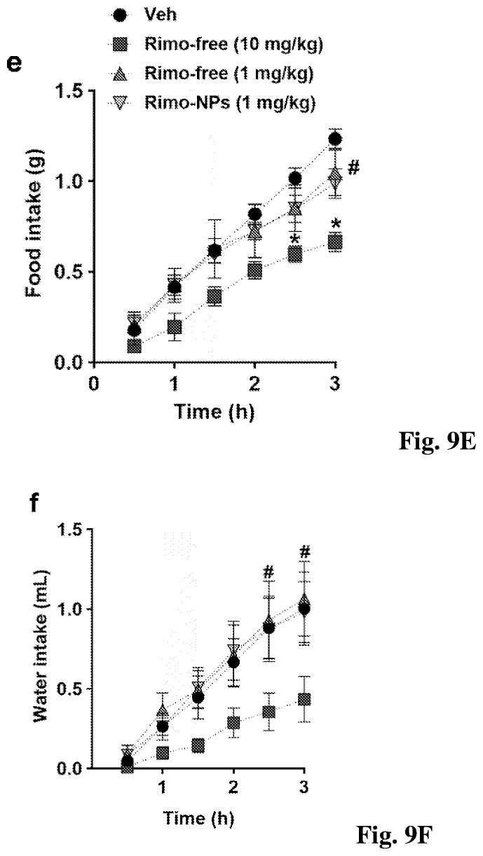

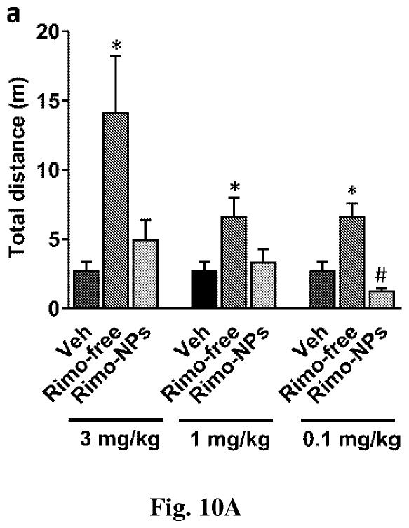

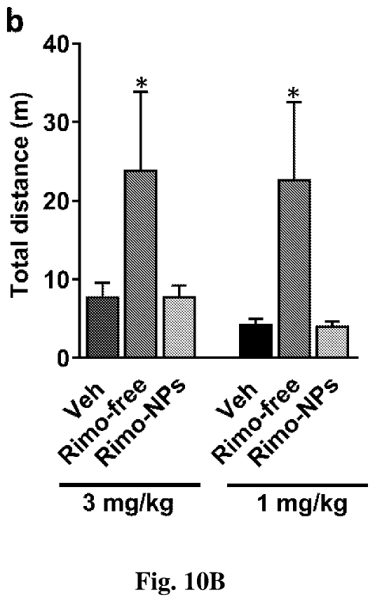

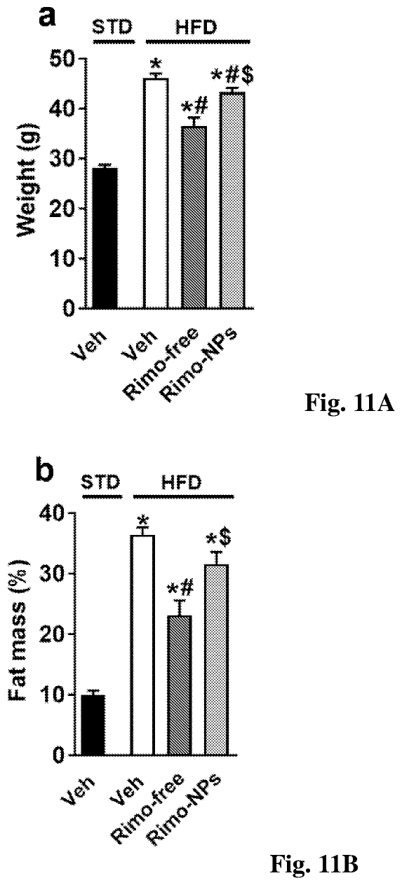

Encouraged by the distinctively different biodistribution of Rimo-NPs vs free rimonabant, we next determined whether the reduced brain levels of Rimo-NPs are associated with reduced behavioral side effects known to be mediated by blocking the brain's CB1Rs. To that end, we used the 1 mg/kg dose for Rimo-NPs, since at this dose, high levels of rimonabant were found in the liver with negligible amounts found in the brain (FIG. 4). In those cases that free rimonabant at 1 mg/kg did not induce a robust effect, we increased the dose to 10 mg/kg and used it as a “positive control” for CNS-mediated side effects. Whereas both ip and iv administrations of free rimonabant induced CNS-mediated hyperactivity, no such effect was found with Rimo-NPs (FIG. 9A). This effect was further validated in a dose-dependent study, demonstrating that only free rimonabant but not Rimo-NPs induced CNS-mediated hyperactivity administrated iv at doses of 0.1, 1, and 3 mg/kg or ip at doses of 1 and 3 mg/kg (FIG. 10). We next assessed the ability of Rimo-NPs to antagonize the brain's CB1R-induced catalepsy. Indeed, only free rimonabant (1 and 10 mg/kg, ip) blocked the cataleptic behavior mediated by WIN-55,212 (FIG. 9B). Interestingly, only the high dose of free rimonabant (10 mg/kg, ip) induced a robust anxiogenic response in the elevated plus maze (EPM) paradigm (FIGS. 9C-D); it also inhibited acute food and water intakes in mice (FIGS. 9E-F). All together, these findings suggest that the CB1R-mediated side effects of rimonabant are CNS-induced in a dose-dependent manner.

### Inability of Rimo-NPs to Affect the Centrally Mediated Regulation of Body Weight and Metabolic Homeostasis

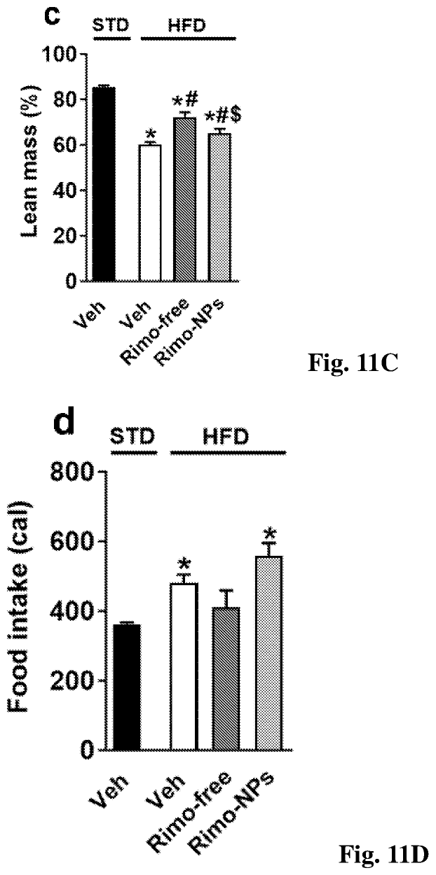

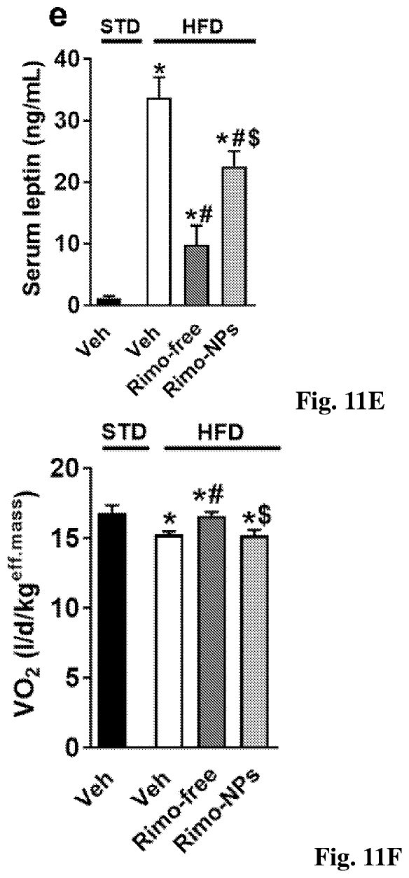

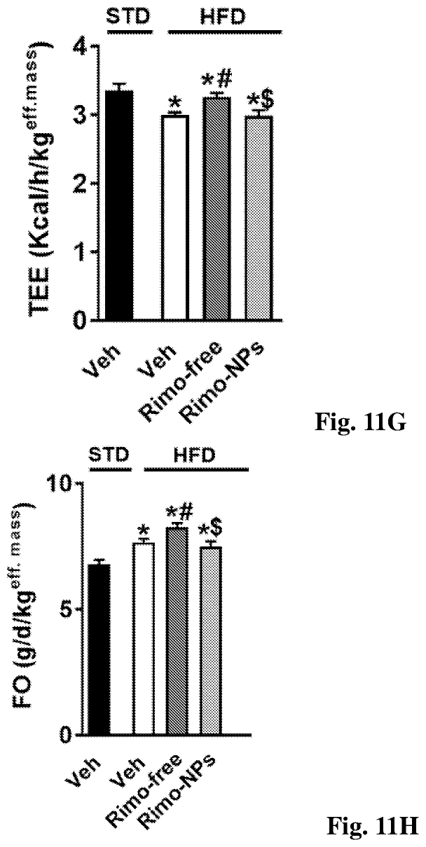

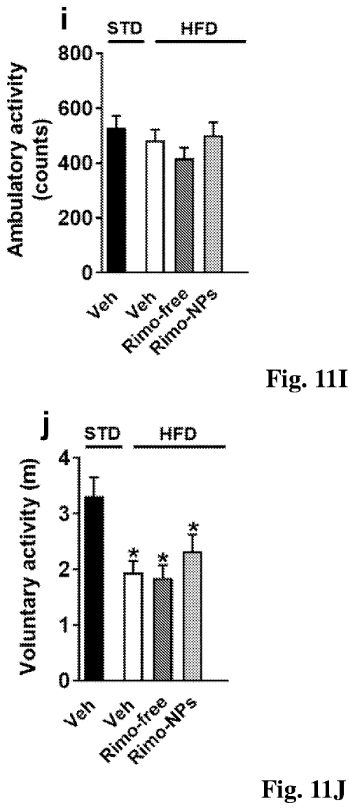

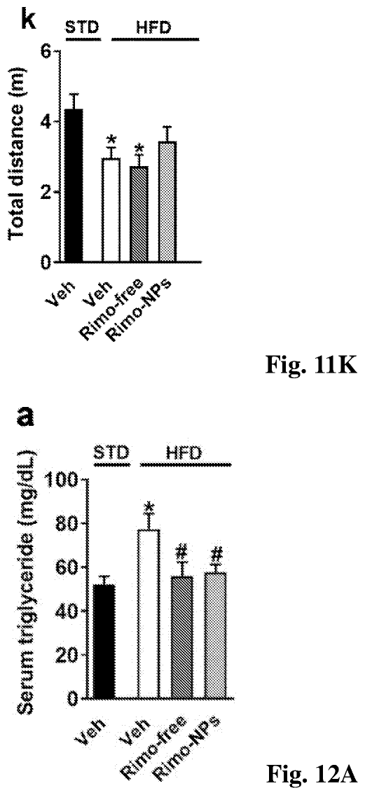

The metabolic effects of Rimo-NPs and free rimonabant were examined in a diet-induced obesity (DIO) mouse model (male C57BL/6J) fed with a high-fat diet (HFD) for 14 weeks. The obese mice were treated daily with Rimo-NPs in comparison to empty NPs (vehicle) and free rimonabant in solution (1 mg/kg/d, ip; for 28 days). Age- and sex-matched mice on a standard diet (STD) served as controls. Following treatment with free rimonabant, and to a lesser extent with Rimo-NPs, the overweightness, increased adiposity, and reduced lean mass were significantly ameliorated (FIGS. 11A-C). The minor effect exhibited by Rimo-NPs is most likely due to the poor penetration of rimonabant into the brain; it was unable to affect the CNS-induced reduction in food intake and to reverse hyperleptinemia (FIGS. 11D-E). In addition, indirect calorimetry assessment at the end of the treatment regimen revealed that free rimonabant treatment, but not by Rimo-NPs, resulted in upregulation of oxygen consumption (VO₂), total energy expenditure (TEE), and fat oxidation (FO) (FIGS. 11F-H), without affecting the animal's activity profile (FIG. 11I-K). This elucidates the notion that the modest improvement in body weight by free rimonabant is most likely due to the centrally mediated increased lipid oxidation.

### Weight-Independent Effects of Rimo-NPs in Ameliorating Obesity-Induced Dyslipidemia, Hepatic Steatosis, and Insulin Resistance

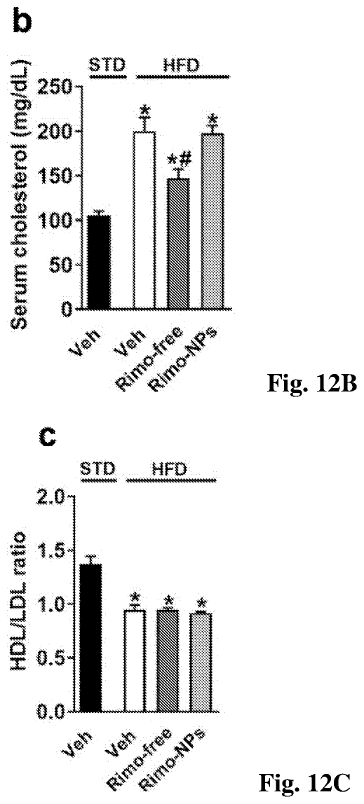

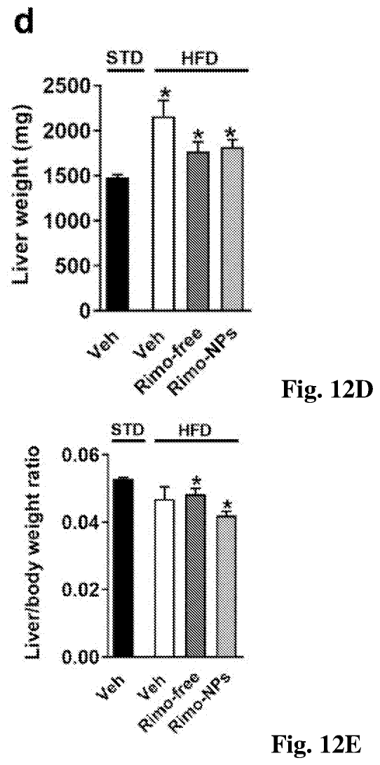

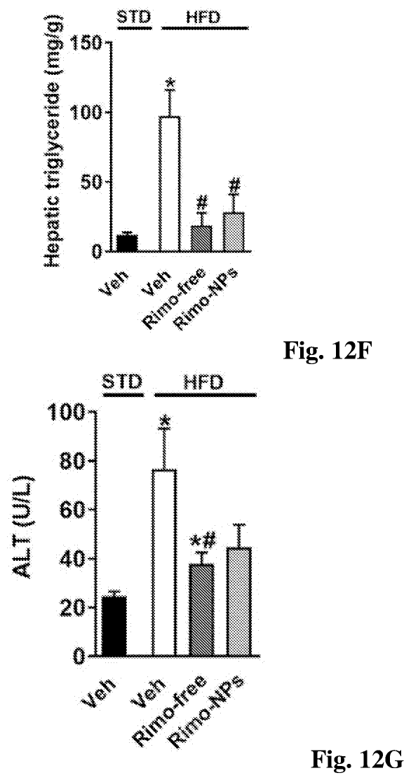

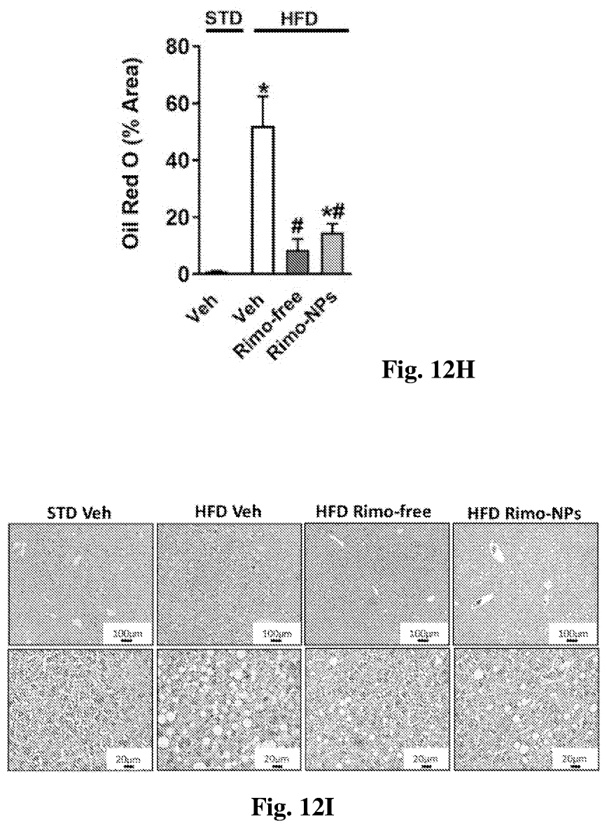

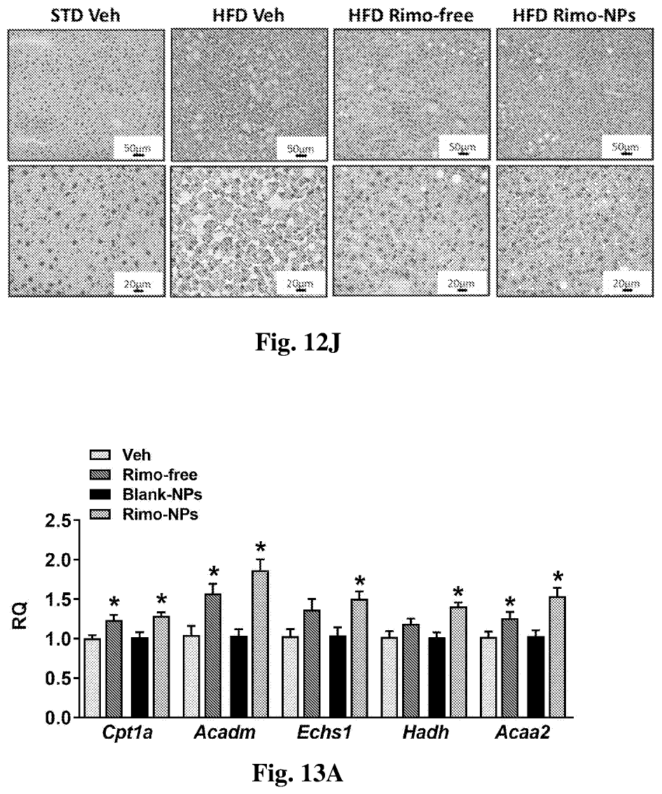

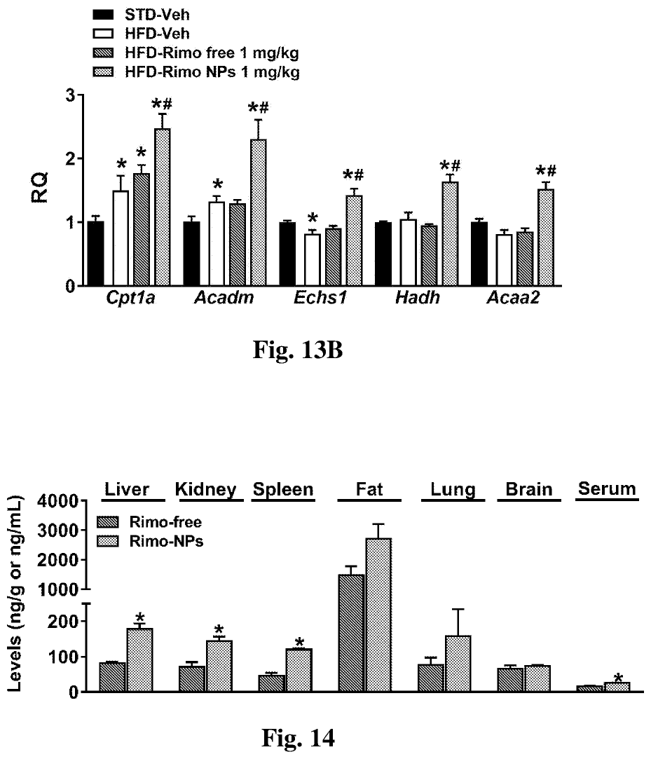

We deciphered the effect of chronic treatment with Rimo-NPs, in comparison to free rimonabant, on obesity-induced dyslipidemia, hepatic steatosis, and insulin resistance. Our findings show that hypertriglyceridemia, but not hypercholesterolemia, was ameliorated by Rimo-NP treatment (FIGS. 12A-B). However, neither of the formulations affected the HDL/LDL cholesterol ratio (FIG. 12C). On the other hand, the HFD-induced fatty liver, reflected by increased liver weight, elevated hepatic triglyceride content, hepatocyte ballooning, fat accumulation, and hepatocellular damage (manifested by the elevated serum ALT levels), were completely reversed by either treatment (FIGS. 12D-J). These positive effects are most likely attributed to the ability of rimonabant to increase hepatic fatty acid utilization/oxidation, as measured acutely in primary mouse hepatocytes exposed to lipotoxic conditions (0.5 mM O:P 2:1) and pre-treated with Rimo-NPs or free rimonabant (FIG. 13), and chronically in DIO mice treated with 1 mg/kg for 28 days (FIG. 13). Interestingly, in the acute condition both treatments significantly upregulated the expression levels of genes associated with fatty oxidation (Cpta1, Acadm, Echs1, Hadh, and Acaa2). However, only Rimo-NPs were found to enhance the expression of these genes in vivo, an effect that could be linked to the higher hepatic exposure of rimonabant found in these animals following chronic administration (FIG. 14).

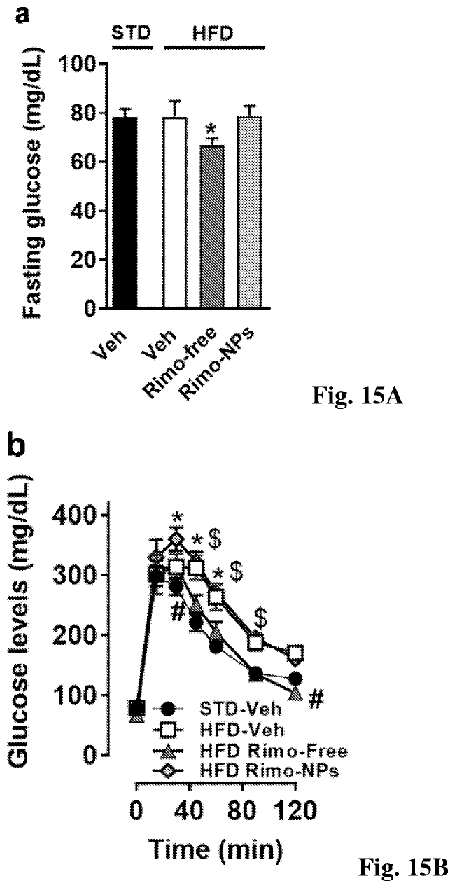

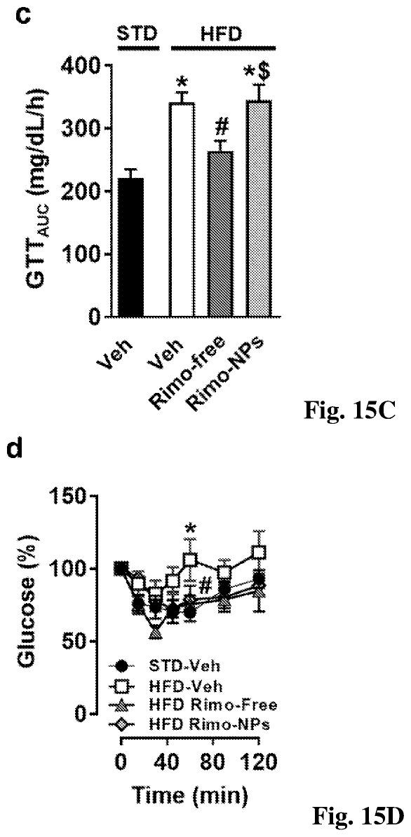

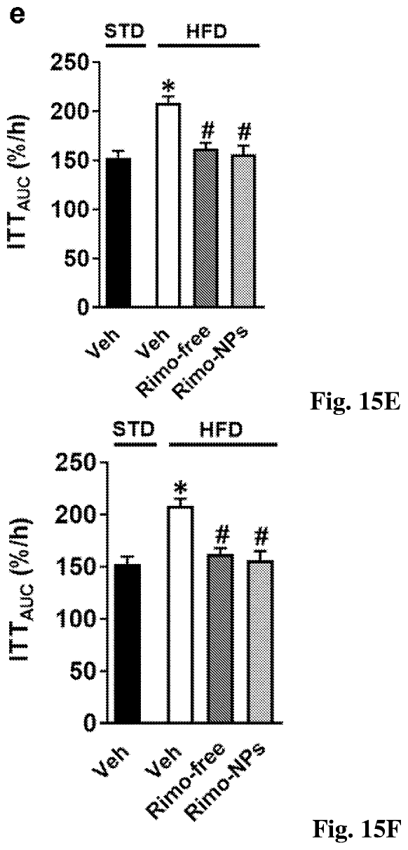

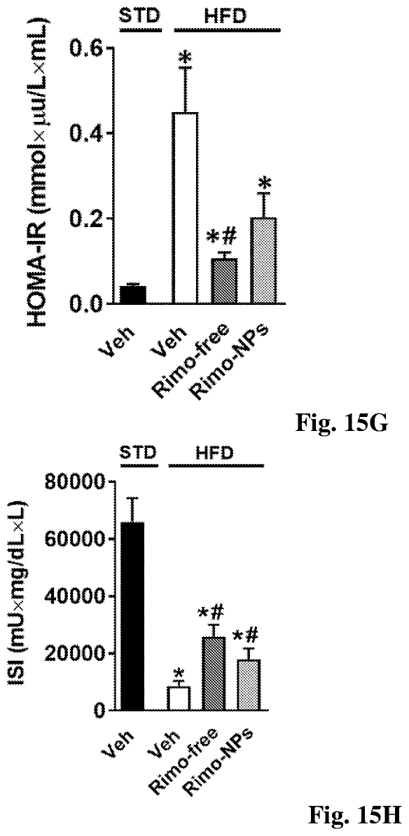

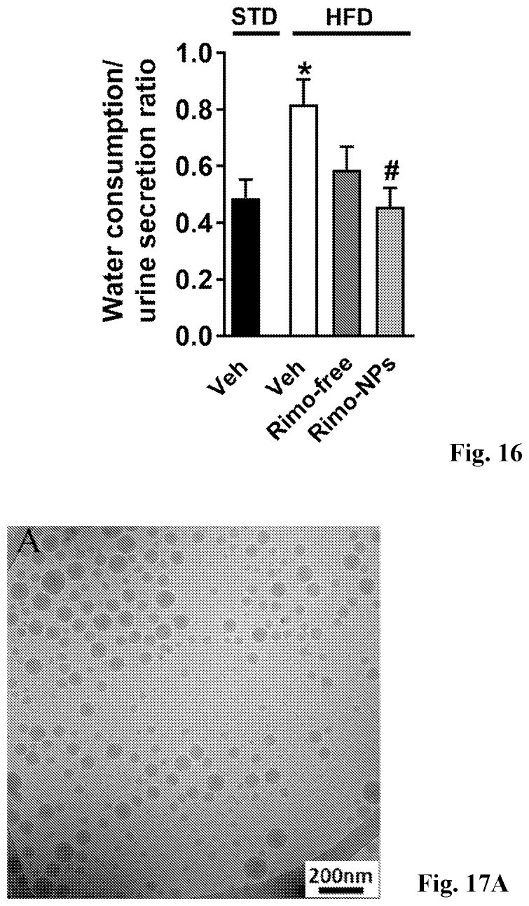

Free rimonabant treatment resulted in both improved obesity-induced glucose intolerance (FIGS. 15A-C), and improved insulin sensitivity (FIG. 15D-H). In contrast, treatment with Rimo-NPs affected only insulin sensitivity, reflected by the reduced glucose levels, following a bolus of insulin, reduced hyperinsulinemia, and improved HOMA-IR and ISI levels (FIGS. 15D-H). In addition, the extensive accumulation of Rimo-NPs in the kidney (FIG. 4) could explain the greater effect of normalizing the water consumption-to-urine excretion ratio (FIG. 16). Congruently with the metabolic efficacy of Rimo-NPs, the levels of rimonabant measured 18 hr following the last injection of the novel formulation, in comparison to free rimonabant treatment, to HFD-fed mice were significantly higher in liver, kidney, spleen, and blood (with a similar trend found in fat and lungs, which did not reach statistical significance). Comparable low levels of rimonabant were found in the brain in the two treatment groups (FIG. 14). These findings further support the peripheral diversion of rimonabant by the NPs, resulting in improved metabolic abnormalities associated with obesity.

### DISCUSSION

Mounting evidence supports CB₁R antagonism as a key pharmacological mechanism to block the overactivation of the eCB signaling system for the treatment of obesity and its cardiometabolic complications, such as NAFLD and T2D. Consequently, the observation that the eCB/CB₁R system is overactive in humans with obesity and in genetic- and diet-induced obese animals led to preclinical developments and clinical efforts to test CB₁R antagonists for the treatment of the metabolic syndrome. Importantly, the first-in-class synthetic CB₁R inverse agonist, rimonabant, was found to reduce weight gain and food intake in a dose-dependent manner under both fasting and non-fasting conditions as well as to inhibit the motivation for palatable food. These data, together with the fact that CB₁R knockout mice are hypophagic and lean, promoted the clinical testing of rimonabant in humans. Indeed, when it was tested in obese/overweight individuals with the metabolic syndrome, rimonabant was found to effectively reduce food intake and body weight, reverse obesity-induced insulin and leptin resistance, decrease hepatic steatosis and liver injury, and improve glucose homeostasis and hyperlipidemias. These findings led to its clinical approval by the European Medicines Agency (EMA) in 2006, under the name of Acomplia® (Sanofi-Aventis), for the treatment of obesity and its related metabolic risk factors in non-diabetic and diabetic overweight and obese patients. However, growing evidence of anxiety, depression, and suicidal ideation, reported in a small but significant portion of individuals treated with rimonabant, led to its eventual withdrawal from the market in 2009. Prompted by the risk of these CNS-mediated adverse effects, several pharmaceutical companies that had been developing proprietary CB₁R blockers terminated their ongoing clinical trials. Moreover, concerns gave been raised regarding the therapeutic potential of this class of molecules in modulating the eCB/CB₁R signaling system for the treatment of obesity and its metabolic abnormalities.

The lack of effective medications for treating the metabolic syndrome in general, on one hand, and the key physiological and pathological roles that the eCB/CB₁R system plays, on the other hand, as well as evidence on the specific deletions of CB₁R in the liver, adipose tissue, kidney, pancreas, and skeletal muscle, have underscored its importance in modulating peripheral metabolic function. Therefore, several novel strategies have been suggested and explored to mitigate/eliminate the CB₁R-induced CNS psychiatric side effects, while retaining the therapeutic benefit of CB₁R blockade. These approaches include developing CB₁R neutral antagonists (e.g., PIMSR, AM4113, and NESS06SM), peripherally restricted CB₁R antagonists (e.g., AM6545, JD5037, MRI-1867, and TM-38837), CB₁R allosteric modulators (e.g., Org27569, PSCNABM-1, and Pepcans), as well as monoclonal CB₁R antibodies (Nimacimab and IM-10). Here, we describe an alternative strategy in which a novel drug delivery system was used as a tool for the peripheralization of rimonabant for the treatment of NAFLD and T2D. We hypothesized that encapsulating rimonabant in NPs for its hepatic distribution would allow us to create the “next generation” of drugs that would target the CB₁R receptor only in the liver, without the side effects associated with blocking the same receptor in the brain. Indeed, by using a nano-drug delivery system, we were able to successfully divert the centrally acting and water-insoluble CB₁R blocker, rimonabant, to the liver, reducing its toxic centrally mediated side effects. In this study, we demonstrated its therapeutic potential to reduce obesity-induced hepatic steatosis and liver injury, improve insulin sensitivity, and reverse hypertriglyceridemia.

As a proof-of-concept for a nano-scaled drug delivery system, we experimented with various formulations. Unfortunately, in our preliminary trials, including use of various polymers, lipids, and surface-active agents, as well as several different methods of NP formulation, we were unable to efficiently entrap rimonabant in nano-drug delivery systems and/or to prevent it from penetrating into the brain. These findings are in accordance with a previous study by Esposito and colleagues, who described the encapsulation of rimonabant in a nanostructured lipid carrier for intranasal delivery in order to bypass the BBB and to target brain CB₁Rs. Here, we took a unique approach to limit the brain penetration of rimonabant and retain its peripheral metabolic actions. The single emulsion evaporation approach used resulted in a stable formulation with high loading capacity and encapsulation efficiency of rimonabant in the NPs. The loosely bound molecules on the surface of the NPs were successfully removed by washing the formulation with an albumin solution, since rimonabant efficiently binds to proteins in the blood. The optimal Rimo-NPs exhibited a retarded release of rimonabant, allowing the drug to remain inside the NPs while circulating in the blood, before reaching peripheral tissues. The ultimate fate of all non-targeted nanomedicines is similar, demonstrating enhanced NP uptake by the liver and spleen. In addition, our data suggest that drug release from the NPs takes place in these organs rather than in the circulation during the first hour after administration. This is well correlated with the biodistribution analyses as well as the negligible side effect profile recorded during the first hour following the administration of Rimo-NPs.

Peripheral targeting of CB₁R antagonists without compromising CNS safety is mandatory when considering this approach for the therapeutic benefits. Ideally, the actual drug levels in the brain should be as low as possible, and the ratio of a target organ exposure to brain exposure for improving CNS safety is another important pharmacokinetic parameter to be considered. Our novel nano-drug formulation complies with both of these features, displaying significantly low levels of rimonabant in the brains of mice acutely injected with Rimo-NPs in comparison with free rimonabant, as well as high liver-to-brain ratios (˜50 to 100, measured by LC-MS/MS or fluorescent-labeled NPs). Comparing the levels of rimonabant in the target organs, which revealed dose- and time-dependent accumulation in mouse liver and hepatocytes treated with Rimo-NPs, allowed us to reduce the therapeutic dose of rimonabant to 1 mg/kg, which also contributed to the negligible levels of the drug found in the brain. A similar tissue biodistribution profile was also found upon chronic administration to HFD-fed mice treated for 28 days with the nano formulation. At that dose (administered iv or ip), Rimo-NPs did not cause CNS-mediated side effects. By using the EPM paradigm, we showed that only a high dose of free rimonabant (10 mg/kg), but not free rimonabant or Rimo-NPs at 1 mg/kg, induces a robust anxiogenic response, manifested by the enhanced time that the mice spent in the closed arms and the reduced time that they spent in the open arms of the maze. These findings, together with the inability of Rimo-NPs at 1 mg/kg to induce centrally mediated hyperambulation, to reverse CB₁R-induced cataleptic behavior, and to inhibit fasting-induced food and water intakes, clearly suggest that while entrapped in NPs, rimonabant is unlikely to induce centrally mediated side effects in mice. Indeed, it has been shown that a low-concentration pharmacological treatment with rimonabant coincides with potent antagonism of CB₁R-mediated G protein activation in vivo, whereas high doses of rimonabant are consistent with its inverse agonism profile, which contributes to the existence of brain-induced adverse effects. Interestingly, although rimonabant is known to induce depression in humans, in the experimental trials used thus far to evaluate its ability to mediate depressive-like symptoms have concluded that this side effect cannot be accurately measured in mice. Specifically, Marinho et al showed that free rimonabant at 10 mg/kg did not induce depressive-like symptoms in the Forced Swim Test. Moreover, Gamble-George and colleagues showed that free rimonabant at 3 and 10 mg/kg did not induce despair-like behavior in the Tail Suspension Test. Since Rimo-NPs were given at a dose of 1 mg/kg, the risk of depression-like behaviors induced by the nano formulation are relatively low.

An apparently unexpected observation was the accumulation of the Rimo-NPs in the kidney. In fact, reports on NPs localizing in the kidneys are rare in the literature. The majority of untargeted NPs (less than 200 nm) primarily tend to accumulate in the visceral organs, liver and spleen, either via circulating mononuclear phagocyte system trafficking or via liver fenestrations (approximately 100 nm). Microparticles (with diameters above 1000 nm) often localize in the lungs due to entrapment in pulmonary capillary beds. In a recent study by Williams et al., it was found that the low opsonization potential of the NPs is a critical parameter responsible for kidney accumulation. Therefore, it can be suggested that specific opsonization of the NPs affected their uptake in the kidneys also here. In addition, their decay over a course of 24 h may suggest that the NPs are eliminated via the kidneys. It should be noted that rimonabant was also accumulated in the kidney (˜300-400 ng/g) in parallel with the presence of NPs in the kidney. However, additional work is needed to further clarify this observation.

Underscoring the contribution of central vs. peripheral CB₁R to the development of obesity and its reversal by CB₁R blockers is a key issue in the eCB field. By using a very low dose of rimonabant encapsulated in NPs that do not cross the BBB, we clearly distinguish between weight loss-dependent and -independent mechanisms regulated by CB₁R blockade. Our data indicate that blockade of central CB₁Rs is essential for the anti-obesity and improved metabolic efficiency of rimonabant, which was found effective in reducing body weight, fat mass, hyperleptinemia, and in increasing the total energy expenditure and fat oxidation. These effects, reported previously with higher doses of rimonabant (3 and 10 mg/kg), are most likely mediated via blockade of CB₁R in the CNS; this was reflected in our study by a lower dose of the free drug (1 mg/kg), manifested in high brain exposure. Interestingly, the reduced metabolic effects of Rimo-NPs on body weight, fat mass, and energy utilization are most likely mediated via the combined result of rimonabant's low brain exposure and a reduction in its systemic presence in tissues, such as adipose tissue and the GI tract, which contribute to weight management. On the other hand, our data indicate that blockade of peripheral CB₁R (mainly in the liver), independent of weight loss, is sufficient to ameliorate obesity-induced hepatic steatosis and liver injury, insulin resistance, and hypertriglyceridemia; these effects are known to be mediated via blocking the CB₁R in hepatocytes. These findings support the critical role of hepatic CB₁R in regulating these metabolic parameters, and further suggest that targeting hepatic CB₁R can be considered a valid therapeutic approach for treating NAFLD and T2D.

In light of the above-mentioned role of hepatocyte CB₁R, a recent study by Wang and colleagues raised concerns about the reproducibility of the effects seen in hepatocyte-specific CB₁R-mice, by demonstrating that deletion of CB₁R in hepatocytes did not alter de novo lipogenesis, insulin resistance, or the development of NAFLD in response to an HFD. With knowledge of this discrepancy, one should also consider that besides hepatocytes, CB₁Rs have also been reported in NPCs such as stellate cells, Kupffer cells, hepatic myofibroblasts, and hepatic vascular endothelial cells. Their contribution to hepatic steatosis, insulin resistance, NASH, hepatic fibrosis and cirrhosis is well documented. In line with these findings, submicron-sized NPs (100-1000 nm) are known to be sequestered by both hepatocytes and NPCs, such as Kupffer cells. To elucidate the intrahepatic distribution of Rimo-NPs, we used both FACS analysis on isolated mouse hepatocytes and NPCs, in addition to immunofluorescence. Whereas our data indicate that Rimo-NPs are largely taken up by hepatocytes, a significant, portion of NPs were also taken up by NPCs. These findings may further contribute to the improved metabolic phenotype of mice treated with the nano formulation, supporting our approach. Moreover, Rimo-NPs taken up by Kupffer cells could release the drug blocking CB₁R in adjacent hepatocytes. An additional mechanism could be that even if Rimo-NPs are delivered intracellularly to hepatocytes or NPCs, once released from the NPs, rimonabant could block the receptor in the mitochondria rather than in the cell membrane. It is worth mentioning that the metabolic effects of rimonabant (as the ‘first-in-class’ CB₁R blocker) on hepatic lipid metabolism and insulin resistance have been vastly studies and described earlier by multiple groups, including us. Our current data showing that chronic treatment of obese mice with Rimo-NPs increased the expression levels of fatty-acid β-oxidation genes further support the well-known positive effect of CB₁R blockade on hepatic lipid metabolism. Nevertheless, further work is required to delineate the role of a specific cell type within the liver or by CB₁R located intracellularly within these cells, and to determine the effectiveness of CB₁R blockade in improving hepatic steatosis and the insulin resistance.

### Summary of Formulation Attempts to Encapsulate Rimonabant

The purpose of the study was to identify a suitable nanocarrier that is capable of encapsulating a drug of choice with high loading capacity, and with a high encapsulation efficacy, so that the formulation will exhibit high stability. In addition, the nanocarriers size was selected to be greater than 100 nm in size, and bear a negative or neutral zeta potential, and the suspension should have a small amount of free drug in it. Lastly, the formulation needs to encapsulate the drug in a way that it will not be released into the circulation in the first hour after injection, to prevent brain penetration before the carriers distribute into peripheral organs.

To craft a proper drug delivery system, we used a variety of polymers using different techniques.

Table 2 demonstrates the various methodologies used and various nanocarriers tested.

TABLE 2
Formulation attempts to encapsulate rimonabant.

| | | | | | Encap. | Loading | Size | |
|---|---|---|---|---|---|---|---|---|
| Type of | | Rimonabant | | | efficacy | capacity | and | Brain |
| NPs | Method | Base/Hcl | Ingredients | Surfactant | (%) | (%) | PDI | penetration |
| Polymeric | Nanoprecipitation | Base | PLGA 50:50 70 kDa, | Solutol HS | 44 | 6.7 | 111.4 ± 0.15 | +++ |
| np | | | ester terminated | 15 | | | | |
| | | Base | PLA 23 kDa, ester | Solutol HS | 50 | 7.6 | 117.8 ± 0.11 | +++ |
| | | | terminated | 15 | | | | |
| | | Base | PLGA 50:50 | Solutol HS | 58 | 8.7 | 121.3 ± 0.14 | +++ |
| | | | 100 kDa, ester | 15 | | | | |
| | | | terminated | | | | | |
| | | Base | PLGA 75:25 | Solutol HS | 45 | 6.8 | 135.6 ± 0.11 | +++ |
| | | | 100 kDa, ester | 15 | | | | |
| | | | terminated | | | | | |
| | Single | Base | PLGA 50:50 | 2% PVA | 10 | 1.0 | 229.3 ± 0.10 | N/A |
| | emulsion | | 100 kDa, ester | | | | | |
| | evaporation | | terminated | | | | | |
| | (data after | HCl | PLGA 50:50 | 2% PVA | 32 | 3.2 | 250 ± 0.15 | N/A |
| | albumin wash) | | 100 kDa, ester | | | | | |
| | | | terminated | | | | | |
| | | Base | PLGA 50:50 50 kDa, | 2% PVA | 63 | 6.3 | 245.9 ± 0.10 | N/A |
| | | | acid terminated | | | | | |
| | | HCl | PLGA 50:50 50 kDa, | 2% PVA | 56 | 5.6 | 232 ± 0.13 | + |
| | | | acid terminated | | | | | |
| | Double | Base | PLGA 50:50 | 2% PVA | 6 | 0.6 | N/A | N/A |
| | emulsion | | 100 kDa, ester | | | | | |
| | evaporation | | terminated | | | | | |
| | (data after | HCl | PLGA 50:50 | 2% PVA | 23 | 2.3 | 254.6 ± 0.15 | N/A |
| | albumin wash) | | 100 kDa, ester | | | | | |
| | | | terminated | | | | | |
| | | Base | PLGA 50:50 50 kDa, | 2% PVA | 40 | 4.0 | 269 ± 0.18 | N/A |
| | | | acid terminated | | | | | |
| | | HCl | PLGA 50:50 50 kDa, | 2% PVA | 47 | 4.7 | N/A | N/A |
| | | | acid terminated | | | | | |
| Liposomes | Liposomes | HCl | POPC, cholesterol, | N/A | 89-93 | 1.2 | 130 ± 0.11 | +++ |
| | preparation from | | Phosphatidylglycerol, | | | | | |
| | microemulsion | | DSPE-PEG | | | | | |

PLGA, Poly Lactic-co-Glycolic Acid. Poly(DL-lactide-co-glycolide) 50:50, 75:25-ratio between glycolic to lactic acid in the polymer chains. PLA, Poly Lactic Acid. POPC, 1-palmitoyl-2-oleoyl-sn-glycero-3-phosphocholine. DSPE-PEG, 1, 2-Distearoyl-sn-glycero-3-phosphoethanolamine-Poly(ethylene glycol), PDI, poly dispersity index.

### Nanoprecipitation

NPs were prepared using the interfacial deposition method. PLGA (complementary to 100 mg of various MWs), oleylcysteine amide (OCA) cross-linker (0, 5, 10, 15, and 20 mg), rimonabant base (15 mg), and MCT oil (0, 7.5, 15, and 30 mg) were dissolved in acetone (25 mL). Solutol HS 15 (10-50 mg) was dissolved in water (50 mL). The organic phase was added dropwise to the aqueous phase with stirring using a Hei-TORQUE 200 stirrer (Heidolph). The formulation's volume was reduced to 10 mL by evaporation (25° C.) at reduced pressure on a Rotavapor (Laborota 4000 efficient, Heidolph Instruments).

Briefly, the PLGA polymer was dissolved in different amounts of acetone ranging between 35 mg and 99 mg with the complement of the other ingredients (up to 100 mg): rimonabant, OCA, and MCT oil. The organic phase was poured into an aqueous phase, which contained 0.1%-0.02% w/v Solutol® HS 15. The volume ratio between the organic and aqueous phases was 1:2 v/v. The suspension was stirred at 900 rpm for 20 min, and then acetone was removed by reduced pressure evaporation. For a concentrated formulation, water was also vaporized until the desired final volume (10 mL) was achieved. The NPs were purified by centrifugation (4000 rpm, for 5 min, at 25° C.).

An example of a formulation prepared by the nanoprecipitation method: The acid-terminated 15 kDa PLGA (50:50 LA:GA) was examined for its loading capacity of the drug in the presence of different amounts of OCA. The formulations exhibited a low average diameter (<100 nm) with a low size distribution (PDI<0.1), likely rendering them unsuitable for iv injection because of renal clearance. The zeta value was low (−20 mV), indicating the presence of aggregates that may have formed over time. The maximal amount of rimonabant in the suspension was ˜5%, and it was lower (3.3%) in the nanosphere. In parallel, a huge precipitate formed immediately. The formulations were characterized by low drug stability, i.e., 1-2 days following their preparation, precipitate was already present, indicating their instability and the need for immediate lyophilization. In addition, the percentage of free drug was around 20%.

Optimization of the formulations prepared by nanoprecipitation: To optimize the loading capacity, encapsulation efficacy, and high stability, a few dozen formulations were prepared. Each time, a different parameter was amended, e.g., the type of PLGAs, different molecular weights of PLGAs, the ratio between glycolic and lactic acid (LA:GA), acid-terminated vs. ester-terminated polymer, testing the surfactant effects (OCA; Solutol), and MCT oils.

### a Summary of the Results is Presented Below:

Adding OCA-Increasing the loading capacity of rimonabant in the formulation and the stability of formulation. By adding OCA, an amphiphilic linker molecule, a derivative of oleic acid, is functionalized with a polar thiol group contributed by cysteine. The dual character of this molecule enables anchoring inside the polymeric matrix core via the lipophilic oleyl chain, while the polar group is docked on the surface of NPs, facilitating thiol functionalization of the surface and the subsequent conjugation to any succinimidyl maleimide-activated molecular moiety. The formulations exhibited a larger average diameter (>120 nm) with a low size distribution (PDI<0.15), and a zeta value of −40 mV, indicating stability over time. The formulation was able to load ˜10% rimonabant and remained stable for ˜10 days. However, examination of the supernatant after sedimentation of the NPs showed that the amount of rimonabant in the nanosphere was ˜6% and that the rest (almost 40% of the amount in the suspension) was freely floating in the solution. There was no second sedimentation of rimonabant in the formulation even 10 days following NP preparation.

Types of PLGAs-Increasing the loading capacity and stability. Similarly, increasing the weight of PLGA to 100 kDa, and changing its end to an ester termination resulted in a dramatic increase in the loading capacity of rimonabant to ˜8%. The ester-terminated PLGA, with a higher proportion of lactic acid and a higher MW, increases the hydrophobicity features; this has the potential to more effectively encapsulate the hydrophobic compounds. In a formulation containing an ester-terminated 70 kDa PLGA together with a 23 kDa PLA, there was even more improvement, to ˜10% LC of rimonabant, and a second sediment formed 10 days after the preparation was created.

Adding MCT oil-Increasing stability. MCT oil was added to the formulation to create a hydrophobic core that dissolves the rimonabant in it. Supplementing the formulation with an MCT oil did not affect the loading capacity or the encapsulation efficacy. However, the time until the formation of the second sediment was later compared to formulations without MCT oil. For example, crystal formation appeared 2 days after preparing the ester-terminated 100 kDa PLGA NPs without MCT oil. It took almost 10 days for the crystals to appear in the same formulation containing the MCT oil.

Solutol concentration—Effect on the free drug in the suspension. In all the above-mentioned preparations, a high percentage of free drug remained in the suspension, not in the NPs (˜4 mg per 15 mg). The reason for the increased free drug in the suspension was probably due to the high concentration of the surfactant Solutol, which resulted in dissolving the drug into Solutol micelles. Therefore, the concentration of Solutol was gradually reduced and the encapsulation efficacy was determined. Indeed, decreasing the percentage of Solutol to even 0.1% of the final concentration resulted in a very small amount of free drug in the suspension (˜200 μg per 1 5 mg), with no effect on the sediment formation.

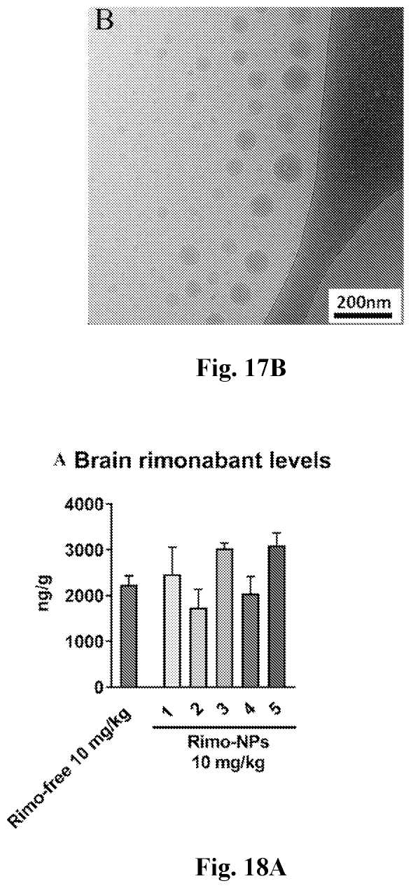

The Cryo-TEM images of the initial formulation (5 mg rimonabant, 90 mg acid-terminated 16 kDa PLGA in 0.5% Solutol) and one of the final optimized formulations (15 mg rimonabant, 65 mg ester-terminated 70 kDa PLGA, 20 mg OCA, and 30 mg MCT oil in 0.1% Solutol) are presented in FIGS. 17A-B.

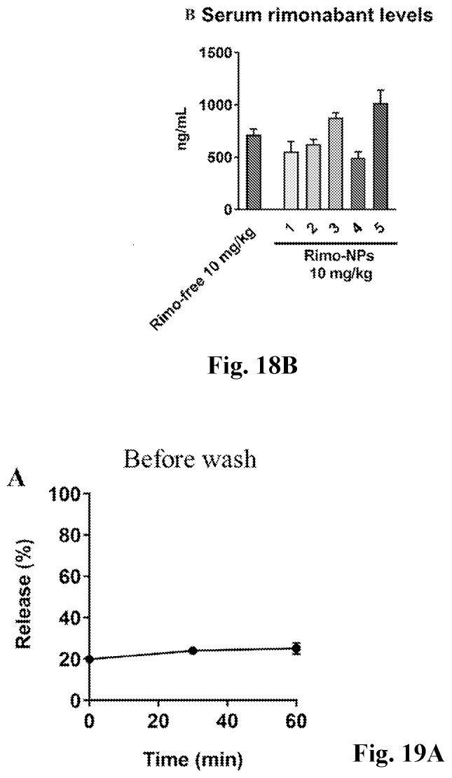

Following the optimization process described above, different formulations (Table 3) were chosen to be iv administered to C57b1/6Jhsd mice, and the brain and serum levels of rimonabant were determined. The formulations were freshly prepared before injection. One hour following administration, the mice were anesthetized, blood was collected, and the mice were perfused with 1×PBS for 1 min to remove the drug from the intravascular space before removing the brain. Brain and serum were extracted, and the drug levels were determined by LC-MS/MS. As shown in FIG. 18, no differences in the penetration of the drug into the brain were found in comparison to the levels of free drug administered at the same dose.

TABLE 3
Formulations prepared by nanoprecipitation for in vivo administration.

| | | Size | | Zeta | Encapsulation | Loading |
|---|---|---|---|---|---|---|
| No. | Ingredients | (d · nm) | DI | (mV) | Efficacy (%) | capacity (%) |
| 1 | 28.75 mg E/T 37 kDa PLGA | 119.3 | 0.074 | −37.7 | 83 | 11.0 |
| | 10 mg OCA | | | | | |
| | 7.5 mg Rimonabant | | | | | |
| | 3.75 mg MCT oil | | | | | |
| 2 | 28.75 mg E/T 75 kDa PLGA | 128.7 | 0.102 | −35.7 | 68 | 9.55 |
| | 75:25 | | | | | |
| | 10 mg OCA | | | | | |
| | 7.5 mg Rimonabant | | | | | |
| | 3.75 mg MCT oil | | | | | |
| 3 | 28.75 mg E/T 70 kDa PLGA | 112.8 | 0.068 | −39.5 | 76.88 | 10.65 |
| | 10 mg OCA | | | | | |
| | 7.5 mg Rimonabant | | | | | |
| | 3.75 mg MCT oil | | | | | |
| 4 | 28.75 mg E/T 37 kDa PLA | 133.3 | 0.077 | −39.7 | 70.46 | 9.44 |
| | 10 mg OCA | | | | | |
| | 7.5 mg Rimonabant | | | | | |
| | 3.75 mg MCT oil | | | | | |
| 5 | 28.75 mg free acid/T 16 kDa | 95.22 | 0.076 | −40.6 | 61.8 | 8.6 |
| | PLGA | | | | | |
| | 10 mg OCA | | | | | |
| | 7.5 mg Rimonabant | | | | | |
| | 3.75 mg MCT oil | | | | | |

### Emulsion Evaporation

The second approach used to encapsulate rimonabant in NPs was the Emulsion Evaporation method.

### Single Emulsion Evaporation

For a typical formulation using the single-emulsion evaporation technique, a solution of 10 mg rimonabant in 200 μL EtOH was added to 3 mL DCM or EtOAc containing 3% PLGA. The solution was added dropwise to 2% PVA in 1×PBS buffer solution (in 25 mL/10 mL for DCM/EtOAc, respectively, 1×PBS buffer) and then sonicated with a microtip probe sonicator (Vibra-Cell tip sonicator, Sonic&Materials, Inc., CT, USA), for 105 s at 80% amplitude over an ice bath, to form a single emulsion (O/W). DCM or EtOAc was eliminated by evaporation under reduced pressure using a rotary evaporator (Buchi, Switzerland), resulting in the formation of NPs. The NPs were recovered by ultracentrifugation (20,000 rpm, for 20 min, at 4° C.), washed twice (with a 10% albumin solution and then with 1×PBS) to remove PVA and unencapsulated rimonabant. The pellet was resuspended in 10% sucrose solution and lyophilized. Dry lyophilized NPs were stored at −20° C. until use.

### Double Emulsion Evaporation

HSA (CSL Behring) solution (2%) was added dropwise (1 mL or 300 μL) under ultra-sonication to a 3 mL EtOAc or DCM solution (respectively) containing 10 mg rimonabant and 90 mg PLGA, and it was sonicated for 105 s at 80% amplitude over an ice bath to form a single emulsion (W/O). Then, the resulting W/O primary emulsions were added dropwise to a 10 mL or 25 mL 2% PVA solution, respectively, and sonicated again for 105 s at 80% amplitude over an ice bath, to form a double emulsion (W/O/W). Next, the organic solvent was eliminated by evaporation under reduced pressure using a rotary evaporator (Buchi, Switzerland), resulting in the formation of NPs.

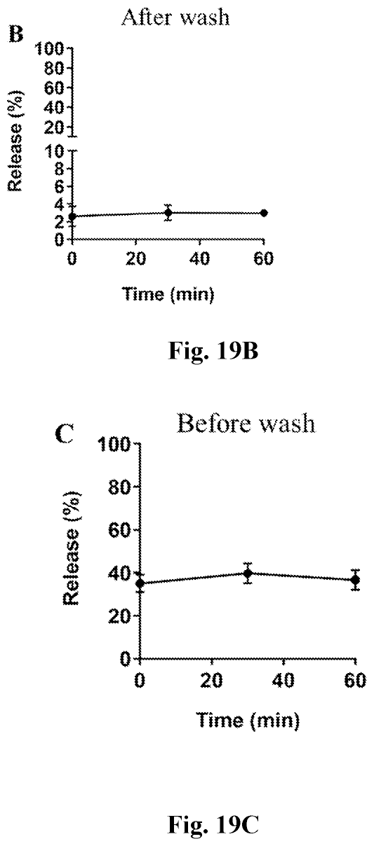

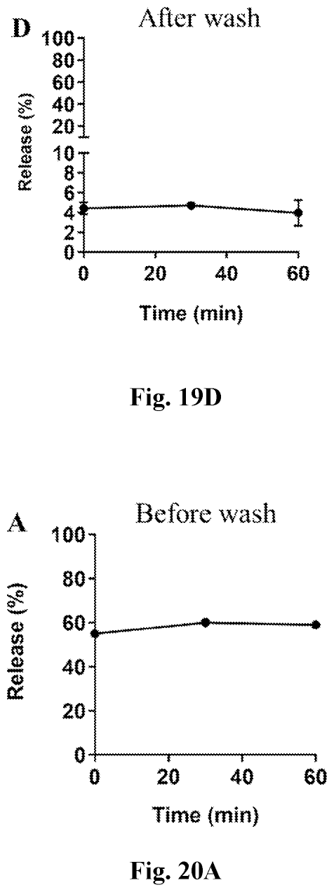

Compared to the nanoprecipitation technique, the single-emulsion evaporation method using rimonabant-base, 16 kDa acid-terminated PLGA and EtOAc resulted in more stable NPs. However, they were too small (˜100 d.nm, PDI 0.19, and zeta-5.44 mV), the content of rimonabant was relatively low (˜6 mg per 10 mg added), and there was a significant amount of free drug in the suspension (>1 mg). Therefore, an attempt was made to encapsulate rimonabant-base in NPs by using the double-emulsion evaporation technique with the same polymer in which the inner phase contained a 2% solution of albumin as a chelator of rimonabant, because rimonabant has a high serum protein binding (>98%), and albumin is a serum protein that exists at 5% in serum. Indeed, the results were promising, demonstrating that only 250 μg rimonabant were found in the external solution, and that larger NPs were obtained (˜220 nm d.nm). The disadvantage was a very high PDI (0.3-0.5). Replacing EtOAc with DCM and using the single emulsion evaporation technique with rimonabant-base and 16 kDa acid-terminated PLGA resulted in an improvement in the formulation in terms of particle size (263.7 d.nm), PDI (0.265), encapsulation efficacy (˜8 mg per 10 mg rimonabant added), and a negligible amount of free drug in the suspension. Moreover, using the double-emulsion evaporation technique with DCM resulted in a narrow range of PDI (0.26 with DCM vs. 0.4 with EtOAc) and a high rimonabant content (8 mg with DCM vs. 6 mg with EtOAc). In attempting to find an explanation for the rapid release of rimonabant, we performed a release test for the two preparations described above (16 kDa acid-terminated PLGA and DCM as a solvent) in 50% human serum in a 1×PBS solution, before and after NPs were washed with a 10% human albumin solution in 1×PBS. The latter step was done to release the drug loosely bound to the surface of the NPs. As shown in FIGS. 19A, C, between 20% and 40% of the drug was rapidly released from the NPs immediately upon exposure to a 50% serum solution. When the NPs were first washed with a 10% albumin solution, most of the rimonabant was released to the albumin solution, leaving a lower amount of the drug in the NPs, which remained encapsulated (FIGS. 19B, D). Data regarding the amount of rimonabant before and after washing the formulation with a 10% albumin solution are presented in Table 4.

TABLE 4
Amounts of rimonabant-base (μg) encapsulated in NPs before
and after washing the NPs with a 10% albumin solution.

| | Single-emulsion | Double-emulsion |
|---|---|---|
| | evaporation | evaporation |
| Before wash | 8,547.5 ± 190.5 | 8,379 ± 469.3 |
| After wash | 3,230 ± 193 | 2,707.32 ± 292.35 |

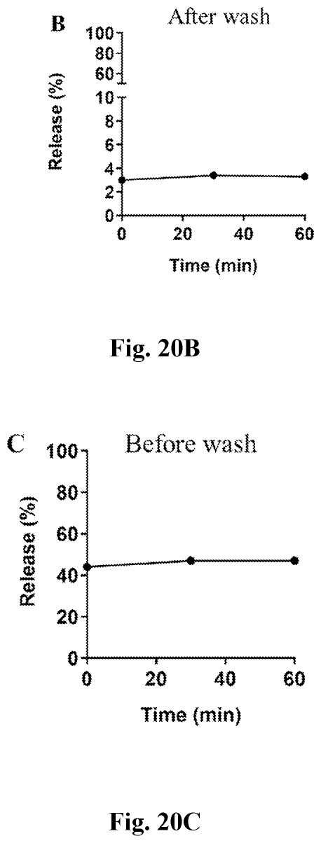

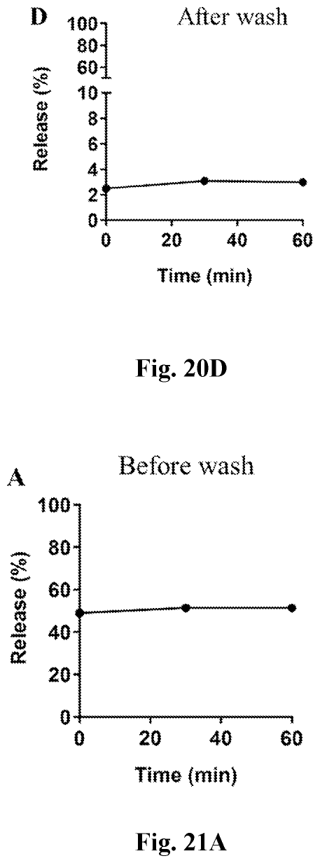

To determine whether the rapid release of rimonabant from the NPs was due to the MW of polymer consisting of the NPs, a new formulation with a different polymer was prepared using the single-emulsion evaporation technique. The polymer chosen was a 100 kDa ester-terminated PLGA LA:GA (50:50), which for single emulsion, resulted in an NP size of 239.5 d.nm, PDI 0.12, an encapsulation efficacy of 67.6%, and a loading capacity of 7.63%. For double emulsion, the encapsulation efficacy was 33.2% and the loading capacity was 3.5%. However, a very rapid release of the drug from the nanoparticles was still observed (FIG. 20). Data regarding the amount of rimonabant in the NPs measured before and after washing the NPs prepared with a 10% albumin solution are presented in Table 5.

TABLE 5
Amounts of rimonabant-base (μg) measured in NPs before
and after washing the NPs with a 10% albumin solution.

| | Single emulsion | Double emulsion |
|---|---|---|
| | evaporation | evaporation |
| Before washing | 7,206 | 3,398 |
| After washing | 1,098 | 599 |

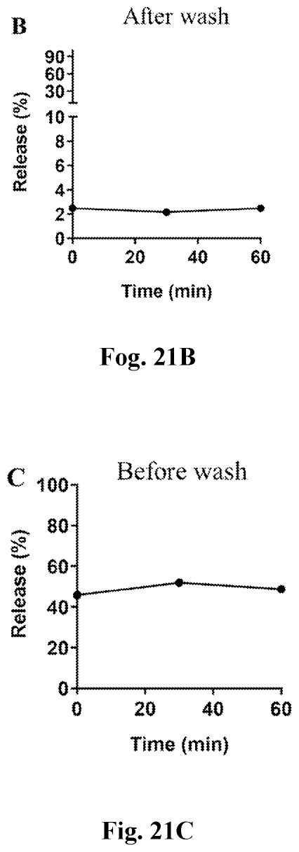

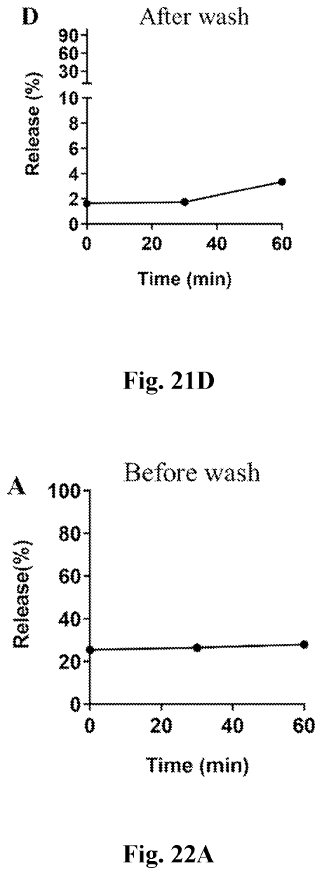

Examination of the release kinetics and the content of rimonabant as HCl, in the NPs (before and after washing the NPs with a 10% albumin solution) revealed no significant improvement in the release kinetics of rimonabant from NPs. However, a much higher amount of rimonabant remained in the NPs after the washing step (for single-emulsion evaporation: 3,156 μg with rimonabant-HCl vs. 1,098 μg with rimonabant-base; for double-emulsion evaporation: 2,343 μg with rimonabant-HCl vs. 599 μg with rimonabant-base, as presented in FIG. 21 and Table 6.

TABLE 6
Amount of rimonabant-HCl (μg) encapsulated in
PLGA 100 kDa ester-terminated NPs before and after
washing the NPs with a 10% albumin solution.

| | Single-emulsion | Double-emulsion |
|---|---|---|
| | evaporation | evaporation |
| Before washing | 8,228 | 7,650 |
| After washing | 3,156 | 2,343 |

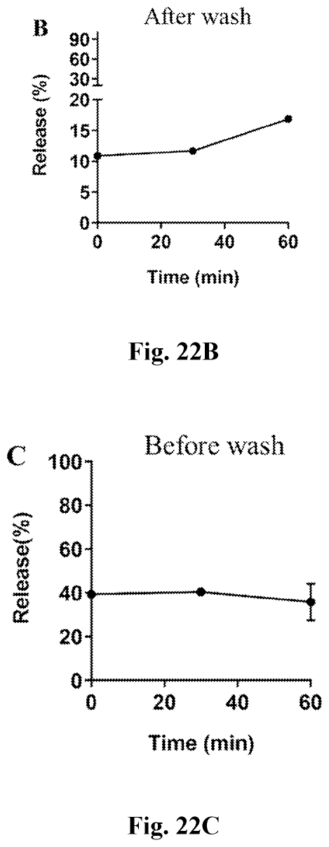

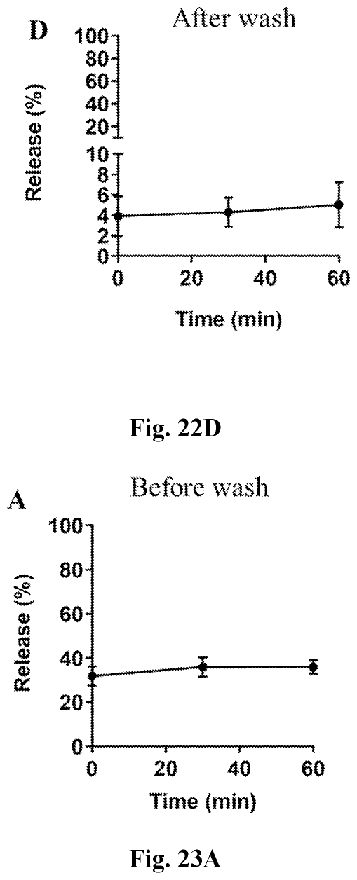

To determine whether acid-terminated PLGA of a higher MW would reduce the rapid release of rimonabant-base from the NPs, a new formulation with a different polymer was prepared using the single- and double-emulsion evaporation techniques. The polymer chosen was an acid-terminated 50 kDa PLGA LA:GA (50:50). Indeed, significant improvements in the release kinetics and in the amount of rimonabant remaining in the NPs were achieved (FIG. 22 and Table 7), suggesting that 50 kDa acid-terminated PLGA is the preferred polymer to use.

TABLE 7
Amounts of rimonabant-base (μg) encapsulated
in PLGA 50 kDa acid-terminated NPs before and
after washing the NPs with a 10% albumin solution.

| | Single-emulsion | Double-emulsion |
|---|---|---|
| | evaporation | evaporation |
| Before washing | 9,264 | 9,893 ± 188.7 |
| After washing | 6,342 | 4,032 ± 268 |

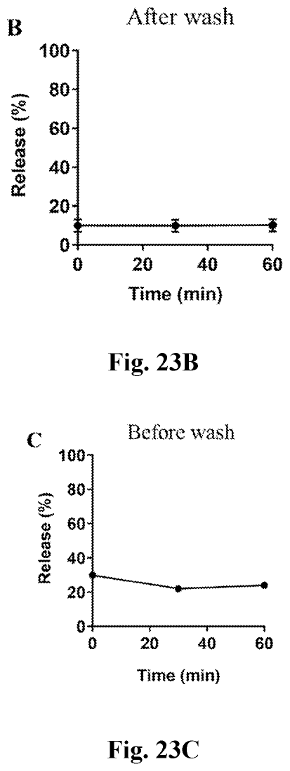

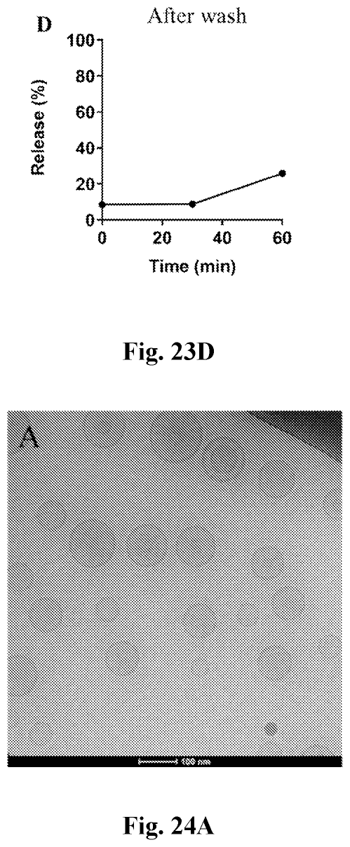

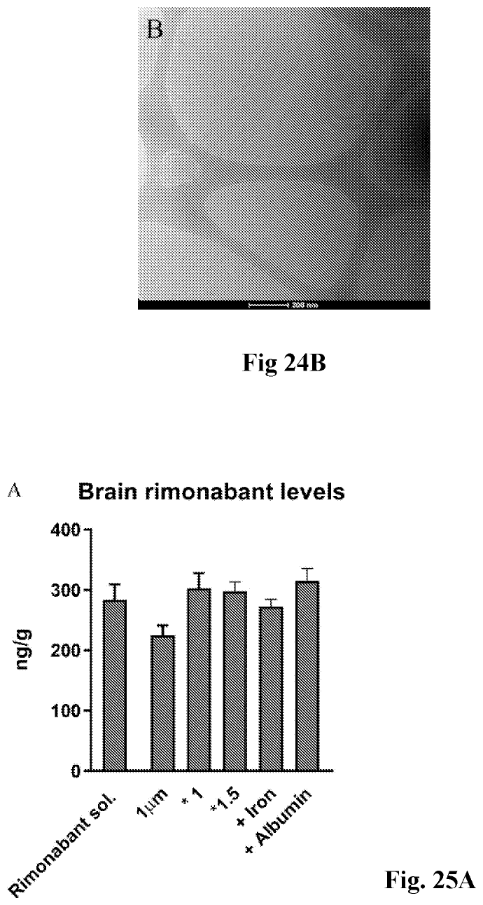

Next, the influence of rimonabant in its acid form (HCl) on its content in NPs and its release kinetics from NPs was examined before and after washing the formulation with a 10% albumin solution. It was found to provide no added advantage over the rimonabant-base (FIG. 23 and Table 8).

TABLE 8
Amounts of rimonabant-HCl (μg) encapsulated
in PLGA 50 kDa acid-terminated NPs before and
after washing the NPs with a 10% albumin solution.

| | Single-emulsion | Double-emulsion |
|---|---|---|
| | evaporation | evaporation |
| Before washing | 9,505.67 ± 411.49 | 8,035 |
| After washing | 4,725.03 ± 851.6 | 4,770 |

The screening tests described above demonstrated that no significant difference exists between single- and double-emulsion evaporation techniques in terms of drug encapsulation efficacy and release kinetics. The preferred polymer for encapsulating rimonabant, as assessed in terms of drug release, and the amount of the drug in NPs after an albumin wash, is 50 kDa acid-terminated PLGA. Moreover, the form of rimonabant should be acidic (HCl).

TABLE 9
Liposomal formulations.

| | | | Loading |
|---|---|---|---|
| | | Size | capacity |
| No. | Ingredients | (d · nm) | (%) |
| 1 | POPC liposomes | 130 | |
| 2 | POPC liposomes (*1.5 POPC) | 130 or 600 | |
| 3 | POPC liposome within Lipidmix (HSPC) | 110 | 17.6 |
| 4 | POPC liposome (x1.5) within Lipidmix | 90 | 11 |
| 5 | POPC liposome (90 nm) within (HSPC/ | 600 | 39.1 |
| | cholesterol) | | |

### Liposome Preparation

POPC (palmitoyl-2-oleoyl-sn-glycero-3-phosphocpalmitoyl-2-oleoyl-sn-glycero-3-phosphocholine) (110 mg), cholesterol (5 mg), PG (egg phosphatidylglycerol) (30 mg), and rimonabant (3 mg) were solubilized in 250 μL EtOH, 250 μL ethyl acetate, and 500 μL 10% sucrose. The solution was heated to 40° C. and lyophilized. Next, the powder was resuspended in water and extruded through polycarbonate filters with defined sizes. The lipids were extruded ten times for 130-140 nm size through filters of 400 nm and 100 nm. The different changes between the formulations are the amount of POPC in comparison to cholesterol, and the inner water phase with or without albumin or iron. The size was between 100 and 600 nm, depending on the shape of the liposomes: regular or multilamellar (Table 9). The multilamellar liposomes were prepared in order to reduce the release of rimonabant from liposomes. The release of rimonabant was very low in 50% FCS in PBS, but when tested on 50% human serum in PBS, the release rate was increased.

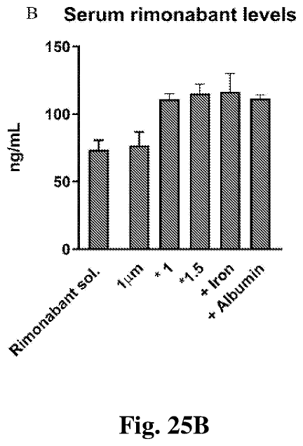

After the optimization process with different ratios of POPC vs. cholesterol, single vs. multilamellar liposomes, and with albumin or iron in the aqua core, the different liposomal formulations were injected iv into male C57Bl/6 mice, and the brain and serum levels of rimonabant were determined by LC-MS/MS. As shown in FIG. 25, no differences in brain penetration of the drug were found, compared to levels of free drug administered at the same dose.

### CONCLUSIONS

The danger posed by NAFLD, as a sign of more serious conditions such as insulin resistance and T2D, is increasingly recognized; however, there is a dearth of pharmacological agents that are effective in the treatment of NAFLD and T2D. Overactivation of the eCB/CB₁R system is thought to play a significant role in the development of obesity and its metabolic abnormalities, such as NAFLD and T2D. Therefore, blockade of CB₁R represents a promising approach to potentially reverse these pathologies. However, the clinical use of globally acting CB₁R blockers (e.g., rimonabant, taranabant, ibipinabant, and otenabant) was halted largely due to their centrally mediated side effects. Whereas several chemical approaches to mitigate brain penetration of these drugs have been recently raised and preclinically validated, a strategy in which a nano-drug delivery system could be utilized to retain the drug's metabolic efficacy without jeopardizing CNS safety has never been reported. Our data show encouraging results for the encapsulation of small amounts of the drug in NPs for therapeutic benefits. When administered to mice in a dose that neither crosses the BBB nor causes side effects, high levels of rimonabant were found in the liver. In addition, the liver-targeted version of rimonabant could ameliorate diet-induced hepatic steatosis as well as insulin resistance in obese mice. This work and its novel findings have important translational/therapeutic implications. Nevertheless, future studies of this unique delivery system should focus on designing an appropriate formulation of these NPs for oral, subcutaneous, or intramuscular delivery to facilitate its practical clinical translation and application.

The invention claimed is:

1.-35. (canceled)

36. A nanocarrier comprising a cannabinoid 1 receptor (CB1R) antagonist, wherein said nanocarrier is configured for peripheralization of the CB1R antagonist contained therein, without inducing a CNS effect, and wherein the nanocarrier is an acid-terminated or an ester terminated PLGA having a molecular weight between 30 and 100 kDa.

37. The nanocarrier according to claim 36, wherein the polymer is a biodegradable polymer having a neutral or a negative surface charge.

38. The nanocarrier according to claim 36, the nanocarrier having a zeta potential between zero and minus 10.

39. A peripherally restricted nanocarrier comprising a central nervous system (CNS) acting CB1R antagonist, for selective modulation of a peripheral CB1R, wherein the nanocarrier is PLGA nanocarrier having a size between 100 and 300 nm and a zeta potential between zero and −10 (minus 10) mV, and wherein the PLGA is an acid-terminated or an ester terminated PLGA having a molecular weight between 30 and 100 kDa.

40. The nanocarrier according to claim 39, wherein the PLGA is of a molecular weight of between 50 and 100 kDa.

41. The nanocarrier according to claim 36, wherein the CB1R antagonist is a neutral antagonist or an inverse agonist of the CB1R receptor having an unencapsulated form capable of crossing the blood-brain barrier and causing CNS mediated side effects.

42. The nanocarrier according to claim 36, wherein the antagonist is an agent capable of effective modulation of a metabolic disease or pathology.

43. The nanocarrier according to claim 42, wherein the metabolic disease is NAFLD.

44. The nanocarrier according to claim 42, wherein the antagonist is an agent capable of effective modulation of a metabolic disease selected from obesity-induced dyslipidemia, hepatic steatosis, liver injury, insulin resistance, reversion of liver weight, elevated hepatic triglyceride content, hepatocyte ballooning, fat accumulation, hepatocellular damage, improvement of insulin sensitivity, reduction of hyperinsulinemia, chronic kidney disease, diabetes, hypertension, and improvement of Homeostatic Model Assessment for Insulin Resistance (HOMA-IR).

45. The nanocarrier according to claim 36, wherein the antagonist is rimonabant or an analogue or isostere or a derivative thereof.

46. A pharmaceutical composition comprising a CB1R antagonist contained in peripherally restricted nanocarriers according to claim 36.

47. A method for treating a metabolically associated disease or pathology, the method comprising administering to a subject in need of such treatment a therapeutically effective amount of a CB1R antagonist contained in peripherally restricted nanocarriers, according to claim 36, wherein the treatment does not induce CNS derived side effects.

48. A method of improving a condition of a subject suffering from a metabolically-associated disease or pathology, without inducing CNS induced side effects, the method comprising administering to said subject a composition comprising nanocarriers containing a CB1R antagonist according to claim 36.

49. A method of peripheralization of a CNS-active CB1R antagonist without inducing CNS-derived side effects, the method comprising containment of said CNS-active CB1R antagonist in nanocarriers and administering said nanocarriers to the subject.

50. A method of treating or preventing a non-alcoholic fatty liver disease (NAFLD) or type 2 diabetes (T2D) or dylipidemia in a subject, the method comprising administering to said subject a composition comprising nanocarriers comprising a CB1R antagonist suitable for treatment or prevention of the NAFLD or T2D, wherein said nanocarrier is configured for peripheralization of the CNS acting CB1R antagonist, without inducing a CNS effect.

51. The method according to claim 50, wherein the CB1R antagonist is a CNS active CB1R antagonist.

52. The method according to claim 47, wherein the metabolically-associated disease or pathology is a non-alcoholic fatty liver disease (NAFLD).

53. A pharmaceutical comprising a plurality of nanocarriers, each containing at least one CB1R antagonist and/or a CNS-active CB1R antagonist, wherein the pharmaceutical composition is an injectable composition suitable for administration to a subject, wherein the nanocarriers are of a size, surface charge and composition selected to prevent the nanocarriers from crossing the brain blood barrier of the subject.

54. The composition according to claim 53, wherein the nanocarriers are characterized by one or more of:
a. the nanocarriers are of an acid- or ester-terminated PLGA;
b. the nanocarriers are of a PLGA having a molecular weight between 30 and 100 kDa;
c. the nanocarrier are of a size between 80 and 100 nm; and
d. the nanocarriers have surface zeta potentials between zero and −10.

55. The composition according to claim 53, wherein the CB1R antagonist is a CNS active CB1R antagonist capable of induing CNS derived side effects when provided in a form other than the nanocarrier.
